(19) United States
(12) Patent Application Publication
(10) Pub. No.: US 2003/0138787 A1
(43) Pub. Date: Jul. 24, 2003

(54) Functional genetic tests of DNA mismatch repair

(76) Inventors: Grant A. Bitter, Agoura, CA (US); Aaron R. Ellison, Thousand Oaks, CA (US)

Correspondence Address: Richard J. Mazza, c/o BitTech, Inc., P.O. Box 1499, Agoura Hills, CA, 91376-1499, US

(21) Appl. No.: 10/109,791

(22) Filed: Mar. 29, 2002

Related U.S. Application Data
(60) Provisional application No. 60/281,636, filed on Apr. 5, 2001.

(51) Int. Cl. C12Q 1/68; C12N 1/18; C12N 15/74

(52) U.S. Cl. 435/6; 435/483; 435/254.2

(57) ABSTRACT

An invention is described which provides a diagnostic approach for diseases, such as HNPCC, that are associated with defects in MMR and provides a method for determining whether any specific genetic sequence of a gene associated with MMR that differs from a consensus sequence is a mutation (i.e., encodes a non-functional protein), a silent polymorphism (i.e., encodes a protein with normal protein function) or an efficiency polymorphism (i.e., encodes a protein with reduced efficiency in MMR). The invention allows the generation of databases of the functional significance of specific amino acid replacements on MMR protein function in vivo, which in turn will allow accurate and unambiguous interpretation of genetic tests of MMR.

### ACKNOWLEDGEMENT

Work taking place in the laboratory when this invention occurred was supported in part by research grants from the National Institutes of Health (R43CA81965, R44CA81965). The U.S. Government may have rights in this invention as a result of this support.

### BACKGROUND OF THE INVENTION

1. Technical Field

Hereditary nonpolyposis colorectal cancer (HNPCC) is an autosomal dominant inherited disease caused by defects in the process of DNA mismatch repair, and mutations in the hMLH1 or hMSH2 genes are responsible for the majority of HNPCC. In addition to clear loss-of-function mutations conferred by nonsense or frameshift alterations in the coding sequence or by splice variants, genetic screening has revealed a large number of missense codons with less obvious functional consequences. The ability to discriminate between a loss-of-function mutation and a silent polymorphism is important for genetic testing for inherited diseases like HNPCC where there exists opportunity for early diagnosis and preventive intervention. The present invention provides quantitative in vivo DNA mismatch repair (MMR) assays in the yeast Saccharomyces cerevisiae to determine the functional significance of amino acid replacements observed in the human population.

2. Background

Colorectal cancer (CRC) is one of the most common cancers, affecting 3-5% of the population in the western world by age 70. Each year approximately 130,000 individuals are diagnosed with CRC and 57,000 individuals die from CRC. Hereditary nonpolyposis colorectal cancer (HNPCC) accounts for approximately 10% of CRC and manifests with a high rate of mortality in the absence of early detection and treatment (reviewed in: (Kinzler & Vogelstein, 1996; Papadopoulos & Lindblom, 1997; Peltomaki & Chapelle, 1997)). Diagnosis of HNPCC in a family is based on kindred analysis using the Amsterdam Criteria (Vasen et al., 1991), which require: i) three or more family members to have had histologically verified CRC, with one being a first-degree relative of the other two, ii) CRC in at least two generations, and iii) at least one individual diagnosed with CRC before age 50. At the molecular level, HNPCC is associated with defects in the cellular process of DNA mismatch repair.

The process of DNA mismatch repair (MMR) corrects non-native DNA structures that form primarily during DNA replication. These aberrant structures include incorrectly paired bases resulting from misincorporation by DNA polymerases, as well as insertion/deletion loops in DNA which form, for example, as a result of microsatellite instability. The amino acid sequences of MMR protein functional domains are conserved from E coli to humans, and the eukaryotic MMR proteins are named based on their homology to E. coli MutS and MutL. Mechanistic studies of MMR in yeast and human cells have elucidated similar processes (reviewed in (Fishel & Wilson, 1997; Jiricny & Nystrom-Lahti, 2000; Kolodner & Marisischky, 1999)). MutSα is a heterodimer of MSH2 and MSH6 while MutSβ is a heterodimer of MSH2 and MSH3. MutSα recognizes base:base mismatches, as well as single base insertion/deletion mispairs. MutSβ also recognizes single base insertion/deletion mispairs but is primarily responsible for recognition of larger insertion/deletion mispairs. Heterodimers of the MutL homologues bind to the MutSα or MutSβ DNA mismatch complex to effect repair. The yeast MLH1-PMS 1 heterodimer (MLH1-PMS2 in humans) binds both MutSα and MutSβ while the yeast MLH1-MLH3 complex (MLH1-PMS1 in humans) binds MutSβ (reviewed in (Kolodner & Marisischky, 1999)).

HNPCC has been shown to be caused by mutations in the hMLH1, hMSH2, hPMS1, hPMS2 or hMSH6 genes. To date, more than 240 mutations have been described, and the vast majority occur in either hMLH1 (60%) or hMSH2 (35%). It is probable that the majority of HNPCC is associated with mutations in either hMLH1 or hMSH2 since inactivation of either of these genes results in impaired replication of a broad spectrum of mismatches (single base:base mismatches and both small and large insertion/deletion loops). The most comprehensive database of sequence alterations observed in genes encoding human MMR proteins and implicated in HNPCC is the International Collaborative Group (ICG) on HNPCC (http://www.nfdht.nl). Additional sequence variants which have been observed also appear in the Human Gene Mutation Database (http://archive.uwcm.ac.uk/uwcm/mg/hgmdo.html) and the Swiss Protein Database (http://www.expasy.ch/sprot/sprot-top.html) as well as several single nucleotide polymorphism (SNP) databases (http://manuel.nih.gov/egsnp/genelist-all.cfm, http://www.genome.utah.edu/genesnps, http://www.ncbi.nlm.nkh.gov/SNP). In addition to mutations in hMLH1 and hMSH2, it has been reported that defects in MMR can be caused by gene silencing due to hypermethylation (Herman et al., 1998). Genetic testing of individuals in HNPCC kindreds should decrease cancer-associated morbidity and mortality in this group. Removal of pre-cancer polyps observed during colonoscopy is highly effective in preventing progression of nonpolyposis colorectal cancer. By identification of those individuals with MMR defects in HNPCC kindreds, routine colonoscopies can be performed with, and restricted to, those individuals who will derive benefits from the procedure.

In the genetic analyses of HNPCC kindreds, more than 25% of the gene alterations observed are minor variants such as amino acid replacements or small in-frame deletions. These sequence variants, furthermore, are scattered throughout the gene coding region. If an observed amino acid replacement can be shown to segregate with disease in the affected family, it suggests, but does not prove, that the amino acid replacement is an inactivating mutation. Frequently, however, small family size or unavailability of clinical samples has precluded attempts to correlate the amino acid replacement with pathogenic effect. As genetic analyses of HNPCC kindreds has continued, an increasing number of minor variants have been documented. To date, missense codons resulting in 86 different amino acid replacements have been described in hMLH1 while 66 have been reported in hMSH2. It is now generally acknowledged (Jiricny & Nystrom-Lahti, 2000; Kolodner, 2000; Peltomaki et al., 1997) that accurate and effective genetic testing for FINPCC will require determination of the functional significance of these minor variants, since the utility of genetic tests is severely compromised if there is any ambiguity in the results.

### SUMMARY OF THE INVENTION

The investigations leading to the present invention demonstrate the usefulness of measuring the function of MMR proteins in vivo. Specifically, a quantitative in vivo assay of DNA mismatch repair has been developed in the lower eukaryote Saccharomyces cerevisiae. This analysis system has been shown to be capable of distinguishing silent polymorphisms from “mutations” (i.e., functionally inactivating variants). This yeast system has now been adapted to provide information that allows the analysis of the function of human MMR protein variants and the elucidation of the significance of the codon changes observed in human genes. The information generated with this technology will be useful for unambiguous genetic testing for MMR defects. The invention reported here also demonstrates the existence of a novel class of amino acid replacements that result in proteins that are functional in MMR, but at a reduced efficiency relative to the native protein. This novel class of variant MMR proteins is referred to as “efficiency polymorphisms”. Some of these amino acid replacements have been observed in sporadic cancers and suggest that individuals in the general human population may have different efficiencies of DNA mismatch repair due to common polymorphisms. The efficiency polymorphisms discovered in this invention, as well as those that can be identified in the future using this invention, are predictive of individual differences in susceptibility to develop cancer. Individuals in the general population may thus be screened for cancer susceptibility as a result of the current invention.

In the described study delineated further herein, missense codons previously observed in human genes were introduced at the homologous residue in the yeast MLH1 (SEQ ID NO: 1) or MSH2 (SEQ ID NO: 2) genes. Genes which encode functional hybrid human-yeast MLH1 proteins were also constructed, and used to evaluate missense codons at positions which are not conserved between yeast and humans. Three classes of missense codons were thus found: (1) complete loss-of-function, i.e. mutations; (2) variants indistinguishable from wild-type protein, i.e. silent polymorphisms; and (3) functional variants which support MMR at reduced efficiency i.e. efficiency polymorphisms. There is a good correlation between the functional results in yeast and available human clinical data regarding penetrance of the missense codon. The discovery of efficiency polymorphisms, some of which did not appear to be associated with HNPCC, raises the possibility that differences in the efficiency of DNA mismatch repair exist between individuals in the human population due to common polymorphisms, and that such polymorphisms predispose to early onset cancer development.

In brief, the present invention provides a diagnostic approach for diseases, such as HNPCC, that are associated with defects in MMR and provides a method for determining whether any specific genetic sequence of a gene associated with MMR that differs from a consensus sequence is a mutation (i.e., non-functional protein), a silent polymorphism (i.e., normal protein function) or an efficiency polymorphism (i.e., functional protein with reduced efficiency in MMR). The invention enables the generation of databases of the functional significance of specific amino acid replacements on MMR protein function in vivo. Such databases will allow accurate and unambiguous interpretation of genetic tests of MMR.

One aspect of the invention comprises a method for distinguishing efficiency polymorphisms from inactivating mutations and silent polymorphisms in a human gene encoding a protein involved in DNA mismatch repair, comprising:

expressing a test genetic sequence in a null yeast host in which the native yeast DNA mismatch repair gene which is the orthologue of said human gene has been deleted or modified to inactivate its function in DNA mismatch repair, and

comparing the mutation rate of a reporter gene in the null yeast host of the previous step with the mutation rate of the same reporter gene in (1) the null yeast host which has not been transformed to express said test genetic sequence and (2) the null yeast host which has been transformed to express the yeast orthologue of said human gene, or a hybrid human-yeast orthologue of said human gene, in order to determine if the test genetic sequence is an efficiency polymorphism.

Another aspect of the invention comprises a method for identifying defects in genes involved in DNA mismatch repair, comprising:

producing by random mutagenesis or in vitro mutagenic DNA synthesis a pool of mutagenized DNA molecules corresponding to said gene,

expressing said pool of DNA molecules in yeast host cells,

growing the yeast host cells, and

screening for yeast clones which exhibit a deficiency in DNA mismatch repair.

In these methods, the test genetic sequence can be a yeast orthologue variant of the human gene sequence or a human-yeast hybrid sequence of said variant. Illustratively, the human gene involved in DNA mismatch repair can be selected from the group consisting of the hMSH2 (SEQ ID NO: 3), hMSH3 (SEQ ID NO: 4), hMSH4 (SEQ ID NO: 5), hMSH6 (SEQ ID NO: 6), hMLH1 (SEQ ID NO: 7), hMLH3 (SEQ ID NO: 8), hPMS1 (SEQ ID NO: 9) and hPMS2 (SEQ ID NO: 10) genes, and especially, the hMLH1 and hMSH2 genes.

Still another facet of the invention comprises a method for determining whether a genetic sequence in an individual is associated with a defect in DNA mismatch repair, comprising comparing said genetic sequence with a genetic information database that has been compiled by use of either of the foregoing methods.

Any of these methods can be used in a diagnostic test setting to evaluate predisposition to the onset of cancer in a human subject.

An additional feature of this invention includes DNA molecules encoding a yeast proteins involved in DNA mismatch repair, in which a portion of the coding sequence has been replaced with the homologous coding sequence of the human orthologue to produce a hybrid human-yeast gene, such that the protein expression product of the hybrid gene retains function in DNA mismatch repair in vivo.

Still another feature of this invention encompasses efficiency polymorphisms, loss-of-function mutations and silent polymorphisms which are variants of DNA mismatch repair genes identified by use of any of the above mentioned methods, including but not limited to variants of the hMSH2, hMSH3, hMSH4, hMSH6, hMLH1, hMLH3, hPMS1 and hPMS2 genes.

### BRIEF DESCRIPTION OF THE FIGURES

FIG. 1. This figure shows the alignment of MLH1polypeptides. Amino acid sequences from human (hs, H. sapien; SEQ ID NO: 11), mouse (m, M. musculus, SEQ ID NO: 12), rat (m, R. norvegicus; SEQ ID NO: 13), fruit fly (dm, D. melanogaster; SEQ ID NO: 14), yeast (sc, S. cerevisiae; SEQ ID NO: 15 and sp, S. pombe; SEQ ID NO: 16), plant (at, A. thaliana, SEQ ID NO: 17), flatworm (ce, C. elegans, SEQ ID NO: 18), and bacteria (sa, S. aureus, SEQ ID NO: 19 and ec, E. coli; SEQ ID NO: 20) were aligned using BLAST algorithms (Altschul et al., 1997). Shaded amino acid residues are identical to those in the human protein. The human hMLH1 missense mutations examined in this study are noted above the appropriate amino acid residue in the human sequence.

FIG. 2. This figure shows the mutation frequencies conferred by missense codons in the yeast MLH1 gene (SEQ ID NO: 1). Strain YBT24 containing pSH91 was transformed with pMLH1 (Complemented) or the indicated mutant form of pMLH1. Mutator refers to YBT24 containing pSH91 but without a pMLH1 plasmid, while WT refers to strain YBT5-1 containing pSH91 but without a pMLH1 plasmid. Mutation frequencies were determined as described in Example 4. The mutant frequencies are presented as the mean±standard deviation of at least 4 replicate cultures of a single yeast clone that expresses the indicated MLH1 gene alteration. In this and subsequent figures, * * denotes a mutation frequency which is not significantly different from the mutator strain, while * denotes a mutation frequency which is intermediate between the mutator strain and complemented strain. Mean mutation frequencies are: WT, 1.1×10⁻⁵; Mutator, 1.8×10⁻³; Complemented, 2.8×⁻⁵; A41 S (SEQ ID NO: 21), 3.2×10⁻⁵; A41 F (SEQ ID NO: 22), 3.6×10⁻³; G64R (SEQ ID NO: 23), 2.2×10⁻³; T65N (SEQ ID NO: 24), 2.0×10⁻³; E99K (SEQ ID NO: 25), 1.9×10⁻³; I104R (SEQ ID NO: 26), 1.7×10⁻³; T114R (SEQ ID NO: 27), 2.7×10⁻³; R214C (SEQ ID NO: 28), 6.3×10⁻⁵; V2161 (SEQ ID NO: 29), 1.6×10⁻⁵; R265C (SEQ ID NO: 30), 3.8×10⁻⁴; R265H (SEQ ID NO: 31), 1.1×10-4; I326A (SEQ ID NO: 32), 4.6×10⁻⁵; I326V (SEQ ID NO: 33), 1.8×10⁻⁵; Q552L (SEQ ID NO: 34), 1.8×10⁻³; R672P (SEQ ID NO: 35), 3.4×10⁻³; and A694T (SEQ ID NO: 36), 1.7×10⁻⁵.

FIG. 3. This figure shows the mutation frequencies conferred by missense codons in the yeast MSH2 gene (SEQ ID NO: 2). Strain YBT25 containing pSH91 was transformed with pMETc/MSH2 (Complemented) or the indicated mutagenized form of pMETc/MSH2. Mutator refers to YBT25 containing pSH91 but without a pMetc/MSH2 plasmid, while WT refers to strain YBT5-1 containing pSH91 but without a pMetc/MSH2. Mutation frequencies were determined as described in Example 4. The mutant frequencies are presented as the mean±standard deviation of 6 replicate cultures of a single yeast clone that expresses the indicated MSH2 gene alteration. Mean mutation frequencies are: WT, 1. Ox10⁻⁵; Mutator, 2.4×10⁻³; Complemented, 1.4×10⁻⁵; G317D (SEQ ID NO: 37), 2.3×10⁻⁵.

FIGS. 4A-B. This figure illustrates the structure and function of hybrid human-yeast MLH1 proteins. (A) Schematic representation of the seven hybrid MLH1 proteins (SEQ ID NO: 38-44) in comparison to full-length native human (SEQ ID NO: 11) and yeast MLH1 (SEQ ID NO: 15). Portions of the hybrid protein representing human sequences are represented with solid bars. Numbers above each bar indicate the amino acid residue of the human portion of each gene. For hybrid genes where the fusion is within the protein coding region, the number of the flanking yeast residue is also indicated. The MMR defect (normalized to the strain expressing wild-type MLH1; (SEQ ID NO: 1) Materials and Methods) is listed to the right of each protein. (B) Mutation frequencies of yeast strains expresssing native and hybrid MLH1 proteins. Each hybrid gene was expressed in YBT24 containing pSH91 and assayed for MMR activity as described in Example 4. The mutant frequencies are presented as the mean±standard deviation of at least 4 replicate cultures of a single yeast clone that expresses the indicated hybrid protein. Mean mutation frequencies are: WT, 1.1×10⁻⁵; Mutator, 1.9×10⁻³; Complemented, 2.5×10⁻⁵; MLH1_h(1-177) (SEQ ID NO: 38), 1.0×10⁻³; MLH1_h(1-86) (SEQ ID NO: 39), 2.5×10⁻⁴; MLH1_h(41-130) (SEQ ID NO: 40), 2.1×10⁻⁴; MLH1_h(41-86) (SEQ ID NO: 41), 1.2×10⁻⁴; MLH1_h(77-134) (SEQ ID NO: 42), 5.4×10⁻⁵; MLH1_h(498-756) (SEQ ID NO: 43), 1.9×10⁻³; MLH1_h(498-584) (SEQ ID NO: 44), 1.8×10⁻³.

FIG. 5. This figure shows the mutation frequencies conferred by missense codons in human-yeast hybrid MLH1 genes. MLH1 Q62K (SEQ ID NO: 45) and R69K (SEQ ID NO: 46) alterations were made in a plasmid containing MLH1_h(41-86) (SEQ ID NO: 41). MLH1 S93G (SEQ ID NO: 47) was made in a plasmid containing MLH1_h(77-134) (SEQ ID NO: 42). The resulting constructs were introduced into YBT24 containing pSH91 (Mutator) and mutation frequencies determined as described in Example 4. The mutator strain complemented with the parental pMLH1_h(41-86) and pMLH1_h(77-134) constructs served as controls. Mutation frequencies are from a representative experiment and are presented as the mean±standard deviation of 5 replicate cultures of a single yeast clone that expresses the indicated missense alteration. Mean mutation frequencies are: Mutator, 2.1×10⁻³; Q62K (SEQ ID NO: 45), 2.0×10⁻⁴; R69K (SEQ ID NO: 46), 2.2×10⁻⁴; MLH1_h(41-86) (SEQ ID NO: 41), 1.2×10⁻⁴, S93G (SEQ ID NO: 47), 4.2×10⁻⁵, MLH1_h(77-134) (SEQ ID NO: 42), 4.9×10⁻⁵.

FIG. 6. This figure shows the mutation frequencies conferred by missense codons in the yeast MLH1 gene. Strain YBT24 containing pSH91 was transformed with pMLH1 (Complemented) or the indicated variant form of pMLH1. Mutator refers to YBT24 containing pSH91 but without a pMLH1 plasmid, while WT refers to strain YBT5-1 containing pSH91 but without a pMLH1 plasmid. Mutation frequencies were determined as described in Example 4. The mutant frequencies are presented as the mean±standard deviation of at least 4 replicate cultures of a single yeast clone that expresses the indicated MLH1 gene alteration. Mean mutation frequencies are: WT, 1.1×10⁻⁵; Mutator, 1.8×10⁻³; Complemented, 2.8×10⁻⁵; I22F (SEQ ID NO: 48), 2.47×10⁻⁴; I22T (SEQ ID NO: 49), 5.8×10⁻⁵; P25L (SEQ ID NO: 50), 4.09×10⁻⁴; N61S (SEQ ID NO: 51), 3.53×10⁻⁴; T79I (SEQ ID NO: 52), 1.68×10⁻³; K81E (SEQ ID NO: 53), 1.71×10⁻³; A108V (SEQ ID NO: 54), 7.84×10⁻⁴; V216L (SEQ ID NO: 55), 2.6×10⁻⁵; I262-del (SEQ ID NO: 56), 5.4×10⁻⁵; L666R (SEQ ID NO: 57), 3.29×10⁻⁴; P667L (SEQ ID NO: 58), 4.23×10⁻⁴; R672L (SEQ ID NO: 59), 3.6×10⁻⁵; E676D (SEQ ID NO: 60), 1.1×10⁻⁵; H733Y (SEQ ID NO: 61), 6.9×10⁻⁶; L744V (SEQ ID NO: 62), 1.3×10⁻⁵; K764R (SEQ ID NO: 63), 1.2×10⁻⁵; and R768W (SEQ ID NO: 64), 2.47×10⁻³.

### DETAILED DESCRIPTION OF THE INVENTION

The process of MMR is conserved from yeast to humans, as are the amino acid sequences of the protein functional domains. We previously described a standardized and quantitative in vivo assay of MMR in the yeast Saccharomyces cerevisiae which can be used for assessing the functional significance of missense codons (Polaczek et al., 1998). In the present invention, the effect of 20 amino acid replacements on MLH1p (SEQ ID NO: 15) and MSH2p (SEQ ID NO: 65) function were evaluated using a reporter gene which measures the stability of an in-frame (GT)₁₆ tract. Codon changes previously identified in human genes were introduced by site-directed mutagenesis at the homologous codon in the yeast gene and tested for in vivo function in S. cerevisiae. The present invention also demonstrates feasibility of constructing genes which encode hybrid human-yeast proteins that are functional in MMR in vivo. These hybrid genes allow functional assays of variant proteins containing human amino acid replacements at residues that are not conserved in yeast and/or where the equivalent residue in yeast is uncertain. There was a good correlation between the in vivo function of proteins with amino acid replacements and available human clinical data regarding penetrance of the missense codon. In addition to identification of silent polymorphisms and complete loss-of function mutations, it was discovered that certain codon changes in hMLH1 (SEQ ID NO: 7) and hMSH2 (SEQ ID NO: 3) give rise to proteins with a reduced efficiency of MMR. Some of these amino acid replacements occurred in individuals who developed CRC but whose families did not satisfy the criteria of HNPCC. These observations indicate that differences in the efficiency of DNA mismatch repair exist between individuals in the population due to common polymorphisms, and that such polymorphisms may predispose to early onset cancer.

In this invention, the functional significance of 37 different amino acid replacements in MLH1p and MSH2p was determined (SEQ ID NO: 21-37), (SEQ ID NO: 45-64). Codon changes were engineered into the yeast gene based on previous observation of the same amino acid replacement in the human protein and a potential implication of the missense variant in cancer development. Quantitative in vivo assays of DNA mismatch repair allowed classification of the amino acid replacements as either silent polymorphsims, loss-of-function mutations or efficiency polymorphisms. In addition to being causative of HNPCC, defects in MMR have been implicated in various sporadic cancers, including tumors of the colon (Borreson et al., 1995), leukemia and lymphoma (Hangaishi et al., 1997; Lowsky et al., 1997) and precancerous lesions and adenocarcinomas of the stomach (Semba et al., 1996). Therefore, analysis of genes involved in MMR will be useful for both presymptomatic susceptibility screening (1NPCC) as well as characterization of other pre-cancers and sporadic cancers. Data on the functional significance of the numerous missense codons being identified in these genes will be necessary for accurate and unambiguous interpretation of genetic test results.

The quantitative in vivo assay of MMR utilized in this study measured microsatellite instability of a (GT)₁₆ tract (Polaczek et al., 1998). Some of our results have been confirmed by investigators using different reporter genes to assess MMR activity (Table 2). Reversion rates of the hom3-10 allele, which detects—1 frameshift mutations (Marischky et al., 1996), was used to show that an A41 S alteration in yMLH1 (SEQ ID NO: 21) was silent while A4 IF (SEQ ID NO: 22) and G64R (SEQ ID NO: 23) replacements gave rise to mutant proteins (Pang et al., 1997). MLH1p variants with 165N (SEQ ID NO: 24) and Ti 14R (SEQ ID NO: 27) amino acid replacements were confirmed as mutants using reversion of the lys2::InsE-A₁₄ and his 7-2 alleles and forward mutation in to canavanine resistance (Shcherbakova & Kunkel, 1999). Reversion of the lys2::InsE-A₁₄ allele measures—1 frameshifts in a (A)₁₄ tract (Tran et al., 1997) while reversion of the his7-2 allele detects +1 and −2 frameshifts in an (A)₇ tract (Shcherbakova & Kunkel, 1999). The canavanine resistant can1 mutants detect a range of mutations including base substitutions, frameshifts, duplications, deletions, translocations and inversions (Chen & Kolodner, 1999; Marischky et al., 1996). In vivo functional results identical to ours but obtained using different reporter genes is consistent with the role of MLH1p and MSH2p in repairing a broad spectrum of DNA mismatches (Kolodner & Marisischky, 1999).

There is very good agreement between the results of our functional genetic tests (Table 2) and available human clinical data concerning the amino acid replacement (reviewed in (Peltomaki & Chapelle, 1997); see also http://www.nfdht.nl and references therein). Human variants corresponding to the yeast MLH1 A41F (SEQ ID NO: 22), 165N (SEQ ID NO: 24) and I104R (SEQ ID NO: 26) replacements were identified in HNPCC kindreds (families that satisfied the Amsterdam Criteria; (Vasen et al., 1991)) and shown to segregate with disease in these families (Bronner et al., 1994; Buerstedde et al., 1995; Hackman et al., 1997; Leach et al., 1993; Nystrom-Lahti et al., 1996; Tannergard et al., 1995). When these codon changes were introduced to the yeast gene, the variant protein was nonfunctional in MMR (mutant defect equivalent to the non-complemented mlh1Δ null strain), demonstrating these amino acid replacements to be loss-of-function mutations. Human variants corresponding to the yeast MLH1 G64R (SEQ ID NO: 23), Ti 14R (SEQ ID NO: 27), Q552L (SEQ ID NO: 34) and R672P (SEQ ID NO: 35) replacements were identified in HNPCC kindreds (Buerstedde et al., 1995; Hutter et al., 1996; Maliaka et al., 1996; Moslein et al., 1996; Nystrom-Lahti et al., 1996) but no data was available regarding segregation with disease. These amino acid replacements were shown here to be loss-of-function mutations. The human variant corresponding to the yeast MLH1 E99K (SEQ ID NO: 25) alteration was entered as an unpublished observation in the ICG-HNPCC mutation database without clinical information. This alteration is a loss of function mutation (Example 5).

The GenBank entry for human MLH1 has a V at amino acid position 219 while both I and L have been reported as common polymorphisms at this position with a population incidence which ranges from 31 to 83% in different geographic regions (Liu et al., 1995; Moslein et al., 1996; Tannergard et al., 1995). The high incidence in the population and lack of linkage to disease make it likely these alterations are silent polymorphisms. However, until this time no data on the in vivo function of these variant proteins was available. The native yeast gene contains V at this position and we demonstrated that a yeast MLH1protein with a V2161 (SEQ ID NO: 29) alteration retained full MMR function. Thus, an amino acid alteration suspected to be a polymorphism in humans was confirmed as a silent polymorphism in the functional studies reported here.

Four different amino acid substitutions (R214C (SEQ ID NO: 28), R265C (SEQ ID NO: 30), R265H (SEQ ID NO: 31), I326A(SEQ ID NO: 32) resulted in MLH1 proteins that are functional, but at reduced efficiency. That is, the efficiency of DNA mismatch repair in strains expressing these variants is intermediate between the mlh1Δ-null mutant and the null mutant complemented with the wild-type yeast gene. The R265H (SEQ ID NO: 31) variant was observed in an HNPCC family that satisfied the Amsterdam criteria and cosegregated with disease [Viel, 1997 #38]. However, this substitution occurred in the same allele which contained a frameshift and may have segregated with, but been unrelated to, cancer progression. The V326A allele in humans was identified in HNPCC kindreds (Buerstedde et al., 1995; Liu et al., 1996). The yeast gene encodes an I at this position, and substitution of a V is a silent polymporphism (Example 5). Replacement of this amino acid with an A, however, results in a protein with reduced activity. The R265C (SEQ ID NO: 30) variant was reported as a pathogenic mutation in the ICG-HNPCC database, citing unpublished data. The R214C (SEQ ID NO: 28) replacement was reported in two “suspected” HNPCC individuals from separate families that did not satisfy the Amsterdam criteria (Han et al., 1996; Miyaki et al., 1995). The data reported here demonstrate that amino acid replacements can result in partial inactivation of DNA mismatch repair, and such decreased efficiencies of MMR are associated with early onset colon cancer. We refer to these substitutions as efficiency polymorphisms.

The G322D alteration in hMSH2 (SEQ ID NO: 66) was initially identified in a family that satisfied the ICG criteria (Maliaka et al., 1996). A number of subsequent studies, however, identified this change in 1-6% of both sporadic cancers and unaffected controls (Liu et al., 1995; Tomlinson et al., 1997) suggesting that it might be a common polymorphism which cosegregated with another mutation in the HNPCC family. In our experiments, the yeast G317D allele (SEQ ID NO: 37) was functional since the mutation frequency was reduced over 100-fold when the variant was expressed in the msh2Δ-null mutant. This variant, however, appeared to have a slightly reduced efficiency of MMR compared to the wild-type yeast MSH2p (SEQ ID NO: 65). In a previous study (Drotschmann et al., 1999b), the yeast G317D allele partially complemented an msh2Δ-null mutant when expressed from a GAL10 promoter, but did not provide any complementation when expressed from the native MSH2 promoter using the lys2::InsE-A₁₄ reporter gene. Our results and those of Drotschmann, et al. (1999) demonstrate that the G317D allele is an efficiency polymorphism. The in vivo function of this variant, moreover, may be sensitive to the levels of expression.

The finding that the yeast protein with the A694T (SEQ ID NO: 36) replacement has full activity was unexpected. This missense codon was found in 3 individuals from separate HNPCC kindreds, and was reported to segregate with disease in these families (Froggatt et al., 1996). There are four possibilities why a clinical association with disease did not correlate with our functional data. First, there is the possibility that we did not target the correct codon in the yeast gene. In this 26 amino acid region of yeast MLH1p, the alanine represented only one of three amino acids which is perfectly conserved in the human protein. The computer-generated alignment, therefore, may be insufficient to unequivocally assign corresponding amino acids in this region. In this study, we demonstrated that hybrid human-yeast proteins can retain MMR function. Utilization of such functional hybrids in this region of MLH1p will overcome the limitations of computer-generated alignments. A second possibility is that the A694T variant retains the ability to repair mismatches in a (GT) microsatellite but is ineffective in repairing other types of mismatches. Use of reporter genes which measure repair of different mismatch structures can be used to address such possibilities. It was recently reported, using the lys2::InsE-A₁₄ reporter gene, that the A694T MLH1p variant is functional in MMR, is capable of interacting with PMS1p, and does not affect the mutator phenotype observed by overexpression of wild-type MLH1p (Shcherbakova et al., 2001). These results are consistent with our observations and support assigning this amino acid replacement in yeast as a silent polymorphism. Third, it is possible that the sequence alteration in humans functions as a mutation in a way other than effects of the amino acid substitution on protein function. For example, it has been recently demonstrated that some missense codons, as well as translationally silent point mutations, can cause exon skipping during the process of splicing (Liu et al., 2001). Finally, the clinical data may be incomplete or misleading.

In the experiments reported here, all variant genes were analyzed in the same yeast strain, utilizing the same reporter gene and identical gene expression conditions. The only difference in expression of the variant MMR genes was the altered codon. We did not evaluate steady-state protein levels, and thus did not determine whether some alterations affect protein stability. The MLH1 efficiency polymorphisms (R214C (SEQ ID NO: 28), R265C (SEQ ID NO: 30), R265H (SEQ ID NO: 31), 1326A (SEQ ID NO: 32)) were tested in the quantitative MMR assay at temperatures of 25° C. and 35° C. Both the mutation frequencies and MMR defects were identical to those observed at 30° C. (data not shown), suggesting that the decreased efficiency of these variants is not due to decreased protein stability. Irregardless of whether the amino acid replacement directly affects the process of MMR, or indirectly affects MMR through altered stability of the protein, the consequence for the cell is an increased mutation rate.

Our results demonstrate the feasibility of expressing hybrid human-yeast MMR proteins that are functional in vivo. Two hybrids that incorporated large regions from the C-terminal end of human MLH1 were not active in MMR. The C-terminal portion of yeast MLH1 p has been shown to interact with PMS1p (Pang et al., 1997). It is possible that the regions of the human protein (>86 amino acids; Results) that were incorporated into the hybrids do not functionally interact with yeast PMS1p and/or MLH1p. Based on the results with the N-terminal hybrids, it is expected that utilization of different smaller human coding sequences in the C-terminus will result in functional hybrids. Human-yeast hybrid proteins containing N-terminal regions of human MLH1 were functional and were used to determine the effect of missense replacements directly in the human coding sequence. In general, the efficiency of the hybrid protein in DNA mismatch repair was inversely correlated with the length of the human segment. The most efficient hybrid human-yeast MLH1p was functional in MMR at an efficiency within a factor of two of the native yeast protein (Example 7). The potential for making a series of hybrids to examine a wider range of human amino acid variants is now established.

A surprising finding in this study was that certain missense codons do not inactivate protein function but result in lower efficiency of DNA mismatch repair. Some of these amino acid replacements had a weak clinical association with cancer development. The substitutions were observed in individuals that developed CRC but whose families did not satisfy the criteria of HNPCC. These observations raise the intriguing possibility that differences in the efficiency of DNA mismatch repair exist between individuals in the population due to common polymorphisms. Certain polymorphisms could exhibit weak penetrance yet still predispose to cancer development. If true, this would predict a specific, genetically determined difference in susceptibility to cancer development. The systems described in this report will be useful for determining whether such genetic variation in the population exists and is associated with cancer development. Elucidation of such relationships may facilitate the future implementation of appropriate preventive strategies.

Genetic testing of individuals at a high risk to develop certain hereditary conditions is a powerful emerging strategy for the prevention of disease. However a persistent ambiguity will arise regarding the functional significance of missense codons identified by gene sequencing. If sufficient biochemical, clinical and population data are lacking, then it becomes impossible to state with confidence if a sequence variation is pathogenic or simply a natural variation in the human population. Systems for assessing the in vivo significance of amino acid replacements will increase the effectiveness of genetic testing programs.

### DESCRIPTION OF SPECIFIC EMBODIMENTS

The invention is further illustrated by way of the following examples, which are not intended to be limiting.

### EXAMPLE 1

### Materials and Methods

MLH1 and MSH2 Mutations and Polymorphisms

Human hMLH1 and hMSH2 missense codons that were examined in functional assays were previously reported in public databases maintained on-line by the ICG-HNPCC (http://www.nfdhtl.nl), Human Gene Mutation Database (http://www.uwcm.ac.uk) and Swiss-prot (http://Hwww.expasy.ch). The databases, which are partially overlapping, were last examined Dec. 31, 2000. Several other missense codons were also reported in publications and review articles as noted in the text.

Bacterial Strains, Growth Conditions and Plasmid Expression Vectors

E coli strains JM109, DH5a and XL1-Blue (Stratagene, La Jolla, Calif.) were used for construction and amplification of plasmids. Unless otherwise described, standard bacterial growth conditions and gene cloning methods were employed (Maniatis et al., 1989). Yeast centromeric expression vector pMETc contains a HIS3 selectable marker and a multicloning site positioned between the MET25 promoter and CYC1 terminator (p413MET25;(Mumberg et al., 1994)). Plasmid pSH91 is a yeast expression vector carrying the URA3 coding sequence containing an in-frame (GT)₁₆ tract (Strand et al., 1993).

Yeast MLH1 and MSH2 Expression Vectors

Expression of the yeast MSH2 gene (SEQ ID NO: 2) from plasmid pMETc/MSH2 was previously demonstrated to complement msh2 chromosomal mutations (Polaczek et al., 1998). For expression of MLH1p (SEQ ID NO: 15), the MLH1 gene coding region plus 1.5 kb of 5′ flanking DNA was amplified by PCR from S. cerevisiae strain S288C genomic DNA (Promega) with primers SEQ ID NO: 67 and SEQ ID NO: 68. The primers introduced a SacI restriction site 5′ to the MLH1promtoer region and an XhoI restriction site at 3′ to the MLH1 coding sequence. The 3.9-kb PCR product was restricted with SacI and XhoI and cloned between the unique SacI and XhoI sites of pMETc. This construction deleted the MET25 promoter and placed the expression of MLH1 coding sequences under control of the MLH1promoter.

Site-Directed Mutagenesis

Mutations were introduced into MMR genes using the QuikChange Site-Directed Mutagenesis kit (Stratagene, La Jolla, Calif.) following the manufacturer's instructions. The protocol employs multiple rounds of synthesis with PfuI DNA polymerase using plasmid DNA as template, but no amplification of the in vitro product. Templates for the mutagenesis reaction were as follows: a plasmid containing MLH1 for MLH1 variants A41 F (SEQ ID NO: 22), A41 S (SEQ ID NO: 21), G64R (SEQ ID NO: 23), 165N (SEQ ID NO: 24), E99K (SEQ ID NO: 25), 1104R (SEQ ID NO: 26), T114R (SEQ ID NO: 27), R214C (SEQ ID NO: 28), V216I (SEQ ID NO: 29), R265C (SEQ ID NO: 30), R265H (SEQ ID NO: 31), 1326A (SEQ ID NO: 32), 1326V (SEQ ID NO: 33), Q552L (SEQ ID NO: 34), R672P (SEQ ID NO: 35) and A694T (SEQ ID NO: 36); a plasmid containing MSH2 for yMSH2 variant G317D (SEQ ID NO: 37); a plasmid containing MLH1_h(41-86) for hMLH1 variants Q62K (SEQ ID NO: 45) and R69K (SEQ ID NO: 45); and a plasmid containing MLH1_h(77-134) (SEQ ID NO: 42) for hMLH1 variant S93G (SEQ ID NO: 47). Sense and antisense oligonucleotide primers were PAGE-purified and, to facilitate screening for mutant clones, included a silent restriction site change in addition to the desired missense alteration (Table 1). For all mutations, at least three independent mutant clones were tested for function in yeast with identical results. At least one clone that contained the appropriate restriction site alteration was sequenced on both strands over the region of interest to confirm the mutation and verify the native sequence over at least 100 bp on either side of the introduced mutation. The data presented are derived from replicate cultures of a single mutant clone that had been confirmed by DNA sequence analysis.

PCR

Routine PCR was carried out with approximately 3 ng plasmid DNA or 400 ng genomic DNA in reaction mixtures containing 50 mM KCl, 10 mM Tris-HCl (pH 9.0), 2.5 mM MgCl₂, 200 μM each of dNTPs, 0.1% Triton X-100, 1% DMSO, 0.04 U/μl Taq polymerase (Promega, Madison, Wis.), and 0.1 μM forward and reverse primers. PCR conditions were as follows: 2 min at 94° C., followed by 35 cycles of 36 sec at 94° C., 150 sec at 55° C., and 150 sec at 72° C. and finished with a 10 min incubation at 72° C. When high-fidelity PCR was required, such as for the production of DNA fragments for generating hybrid human-yeast genes, Pfu DNA polymerase (Stratagene) and PCR conditions recommended by the manufacturer were used.

DNA Sequencing

DNA sequencing was performed at commercial sequencing facilities using ABI BigDye Terminator chemistry and ABI automated DNA sequencers (models 377 and 3700).

Statistical Analysis

Differences in mutant frequencies between yeast strains were assessed by Anova Multiple Student's T-test (Statview 4.5). P values of <0.05 were considered significant.

### EXAMPLE 2

### Construction of Hybrid Human-Yeast MLH1 Genes

Genes encoding hybrid human-yeast MLH1 proteins were constructed by replacing portions of the yeast MLH1 gene (SEQ ID NO: 1) with the homologous human coding sequence from the hMLH1 gene (SEQ ID NO: 7). The hybrid constructions were designed to maintain the open reading frame and precisely substitute conserved regions between the human and yeast proteins. DNA sequencing was performed to verify correct clones. DNA sequences of the cloned human fragments were found to exactly match the Genbank report (accession #U07418).

Hybrid MLH1-h(1-177) (SEQ ID NO: 38) was constructed by overlap extension PCR using previously described strategies (Bitter, 1998). A 591-bp fragment containing the yeast MLH1 5′ regulatory region (nucleotides −586 to +6 relative to A of translation initiator codon) was amplified from S. cerevisae S288C genomic DNA using primers SEQ ID NO: 109 and SEQ ID NO: 110. Codons 3-177 of human MLH1 were amplified from a hMLH1 cDNA clone (ATCC # 217884; the first two yeast and human amino acids are identical) using primers SEQ ID NO: 111 and SEQ ID NO: 112. A 1.8-kb C-terminal portion of yeast MLH1, including codons 174 to 769, was amplified with primers SEQ ID NO: 113 and SEQ ID NO: 68. Approximately equimolar ratios of the three fragments were combined and amplified with primers SEQ ID NO: 109 and SEQ ID NO: 68. The resulting 2.9-kb overlap extension product was gel purified, digested with AflII and XhoI and cloned into plasmid pMLH1, replacing the native MLH1 gene.

Hybrid MLH1_h(1-86) (SEQ ID NO: 39) was constructed using a 2-piece overlap extension reaction. A fragment of the human hMLH1 (SEQ ID NO: 7) cDNA containing codons 3-86 was amplified by PCR using primers SEQ ID NO: 111 and SEQ ID NO: 114. The 255-bp PCR product was diluted and mixed with an approximately equimolar amount of the 591-bp yeast MLH1 5′ regulatory region fragment (described above) and overlap extension PCR was carried out using primers SEQ ID NO: 109 and SEQ ID NO: 114. The 0.85-kb overlap extension product was digested with AflII and AatII and cloned into plasmid pMLH1, replacing the native yeast MLH1 segment.

Hybrid MLH1_h(41-130) (SEQ ID NO: 40) was constructed by cloning a 285-bp fragment of the human hMLH1 cDNA containing codons 41-130 between the ClaI and NdeI sites in pMLH1 and replacing codons 38-126 of the native yeast MLH1 gene. The human fragment was amplified using primers SEQ ID 115 and SEQ ID 116, which introduce ClaI and NdeI sites, respectively. The PCR product was digested with ClaI and NdeI to allow in-frame cloning into the yeast MLH1 gene which had been subcloned as a SacI-XhoI fragment into pBluescript II (Stratagene). The hybrid MLH1 gene was subsequently recloned into pMLH1, replacing the native yeast gene.

Hybrid MLH1 h(41-86) (SEQ ID NO: 41) was constructed by PCR amplification of a 140-bp fragment containing codons 41-86 of the hMLH1 cDNA and direct cloning into the yeast MLH1 gene between the ClaI and AatII sites. The human segment was amplified with primers SEQ ID NO: 115 and SEQ ID NO: 114, which introduce ClaI and AatII sites, respectively. The PCR product was digested with ClaI and AatI to allow in-frame cloning into the yeast MLH1 gene in expression vector pMLH1.

Hybrid MLH1_h(77-134) (SEQ ID NO: 42) was constructed in a 2-piece overlap extension reaction. A 170-bp fragment of the hMLH1 gene containing codons 77-134 was PCR amplified using primers SEQ ID NO: 117 and SEQ ID NO: 118, which introduces an AatII site at the 5′ end. A 1.9-kb fragment of yeast MLH1 containing codons 132 to 769 was amplified by PCR using primers SEQ ID NO: 119 and SEQ ID NO: 68. The two fragments were gel purified, diluted, mixed in equimolar amounts and amplified using primers SEQ ID NO: 117 and SEQ ID NO: 68. The overlap extension product was digested with AatII and XhoI to allow in-frame cloning into pMLH1, replacing the native yeast MLH1 gene.

Hybrid MLH1_h(498-756) (SEQ ID NO: 43) was constructed by direct cloning of a 829-bp fragment containing codons 498-756 of human hMLH1 into the yeast gene. The human fragment was amplified by PCR using primers SEQ ID NO: 120 and SEQ ID NO: 121. These introduce a Bsu36I site at the 5′ end and an XhoI site at the 3′ end and allow in-frame cloning into the yeast MLH1 gene in pMLH1 as a Bsu36I-XhoI fragment, replacing codons 506 to 769 of the yeast gene.

Hybrid MLH1_h(498-584) (SEQ ID NO: 44) was constructed using a 2-piece overlap extension reaction. A 290-bp fragment of hMLH1 containing codons 498-584 was PCR amplified with primers SEQ ID NO: 120 and SEQ ID NO: 122. A 560-bp fragment of yeast MLH1, containing codons 595-769, was amplified with primers SEQ ID NO: 123 and SEQ ID NO: 68. Approximately equimolar amounts of each fragment were mixed and subjected to overlap extension PCR using primers SEQ ID NO: 44 and SEQ ID NO: 68. The approximately 800-bp product was digested with Bsu36I and XhoI for replacement of the equivalent fragment in pMLH1.

### EXAMPLE 3

### Yeast Strains, Growth Conditions and Transformations

All yeast strains used in this invention are derivatives of S. cerevisae YPH500 which has the genotype MATα ade2-101 his3-Δ200 leu2-Δ1 lys2-801 trp1-A63 ura3-5 (Sikorski & Hieter, 1989). Strain YBT5-1 was described previously (Polaczek et al., 1998) and has the genotype MATα ade2-101 his3-A200 lys2-801 trpl-A63 ura3-52.

Strain YBT24 (MATα ade2-101 his3-A200 leu2-Δ1 lys2-801 trp1-A63 ura3-52 mlh1Δ::LEU2) contains a deletion of the entire MLH1 coding region and was generated by chromosomal targeting using a DNA fragment constructed by overlap extension PCR procedures (Bitter, 1998). Briefly, nucleotides −140 to +6 of yeast MLH1 (SEQ ID NO: 1)) (relative to the A of the translation initiator codon at +1) and nucleotides 2299 (termination codon at 2306) to 2684 were PCR amplified from S. cerevisiae S288C genomic DNA. The yeast LEU2 gene coding region plus 440 bp of 5′ flanking and 40 bp of 3′ flanking DNA was also PCR amplified from S. cerevisiae S288C genomic DNA. The 5′ end of the LEU25′ primer was homologous to the MLH1 upstream 3′ primer while the 5′ end of the MLH1 downstream 5′ primer was homologous to the LEU23′ primer. Approximately equimolar amounts of each PCR product were mixed and subjected to overlap extension PCR using the outermost MLH15′ and 3′ primers. The resulting 2.2-kb MLH15′-LEU2-MLH13′ fusion was transformed into S. cerevisiae strain YPH500 and leucine prototrophs were selected. Genomic DNA was isolated from one clone, strain YBT24, which exhibited a mutator phenotype using the pSH91 reporter gene and confirmed by PCR analysis to have the entire MLH1 coding region deleted and replaced by the LEU2 gene (data not shown). Strain YBT25 (MATα ade2-101 his3-A200 leu2-Δ1, lys2-801 trp1-Δ63 ura3-52 msh2Δ::LEU2) contains a deletion of MSH2 from codon 2 through the termination codon. Generation, selection and confirmation of YBT25 was similar to construction of YBT24 except that a MSH25′-LEU2-MSH23′ overlap extension product was used for gene targeting.

The MMR reporter plasmid pSH91 (Strand et al., 1993) was transformed into strains YBT5-1, YBT24 and YBT25, selecting for tryptophan prototrophs. The strains were maintained in SD medium supplemented with adenine, histidine and lysine. The additional selection for uracil prototrophy maintains the cultures with 100% of the pSH91 containing an in-frame (GT) tract. YBT24 and YBT25 containing pSH91 were also transformed with pMLH1 and pMetc/MSH2, respectively, and maintained on SD medium supplemented with adenine and lysine. Transformations were carried out by the polyethylene glycol-lithium acetate method (Ito et al., 1983). Yeast strains were stored at −80° C. in 15% glycerol.

### EXAMPLE 4

### In Vivo MMR Assay

The standardized in vivo assay for DNA mismatch repair has been described in detail elsewhere (Polaczek et al., 1998). The assay is based on instability of 33-bp (GT)₁₆ tract which is inserted in-frame in the 5′end of the yeast URA3 gene coding region in plasmid pSH91. The (GT)₁₆ microsatellite is unstable during DNA replication and, if insertion/deletion loops are not repaired by MMR, ura3 mutants form due to frameshift mutations. Selection on plates containing 5-fluoroorotic acid (FOA) is used to quantitate ura3 mutants (Boeke et al., 1984). Briefly, yeast strains were grown for 24 hours in SD media (0.67% yeast nitrogen base without amino acids, 2% dextrose) supplemented with adenine and lysine. An additional supplement of histidine was included for strains that did not carry MLH1 or MSH2 expression vectors. An equivalent volume of cells from each saturated culture was subcultured (1:100) into fresh media containing the same supplements as above plus uracil, and grown an additional 24 hours. The presence of uracil allows growth of any newly formed ura3 mutants in this culture. After overnight growth to saturation, aliquots (25 and 100 μl) of each culture were plated on SD plates containing adenine, lysine, uracil and 1 mg/ml FOA to determine the concentration (cells/ml) of ura3 mutants. The concentration (cells/ml) of total viable plasmid containing cells was determined by serial dilution and plating on SD plates supplemented with adenine, lysine and uracil. Additional supplementation with histidine was included if the strain did not carry MLH1 or MSH2 expression vectors. Colonies were counted 2-3 days later. Mutant frequencies were calculated by dividing the concentration of FOA-resistant colonies by the concentration of viable cells. At least four independent cultures of each yeast strain carrying variant MMR genes were assayed. The MMR defect is defined as the ratio of the mutation frequency in the test strain divided by that observed in the same strain complemented with the wild-type yeast gene.

### EXAMPLE 5

### Functional Assay of Mlh1p Variants Containing Amino Acid Replacements

Function of the MMR protein variants was determined using a standardized MMR assay that measures stability of a (GT)₁₆ tract in vivo (Polaczek et al., 1998). For these experiments, we constructed haploid yeast strains that have complete deletions of the chromosomal coding region of MLH1 (strain YBT24) or MSH2 (strain YBT25). Utilizing the reporter gene (GT)₁₆-URA3 (pSH91; Materials and Methods), both YBT24 and YBT25 exhibit a mutation frequency (Materials and Methods) more than 100-fold greater than a MMR wild-type strain. As demonstrated below, the mutation frequency can be reduced to approximately that observed in the wild-type strain by complementing the chromosomal null mutations with a plasmid expressed (Materials and Methods) wild-type yeast MLH1 (strain YBT24) or MSH2 (strain YBT25) gene. The mean mutant frequency in plasmid-complemented chromosomal null mutants was used as the basis for comparing activity of the variant MMR proteins described below. MMR defect is defined as the mutant frequency conferred by the variant protein in the chromosomal null mutant divided by the mutant frequency observed in the chromosomal null mutant expressing the wild-type yeast gene. All MLH1 or MSH2 variants were analyzed in the same host containing the same reporter gene and were expressed from the same expression vector as the wild-type yeast gene. For all mutagenized genes, at least three independent clones were tested for MMR function with identical results. The DNA sequence on both strands of the mutagenized gene was confirmed for one mutant, and this clone was assayed in replicate cultures (n>4) for determination of the mutation frequencies reported below.

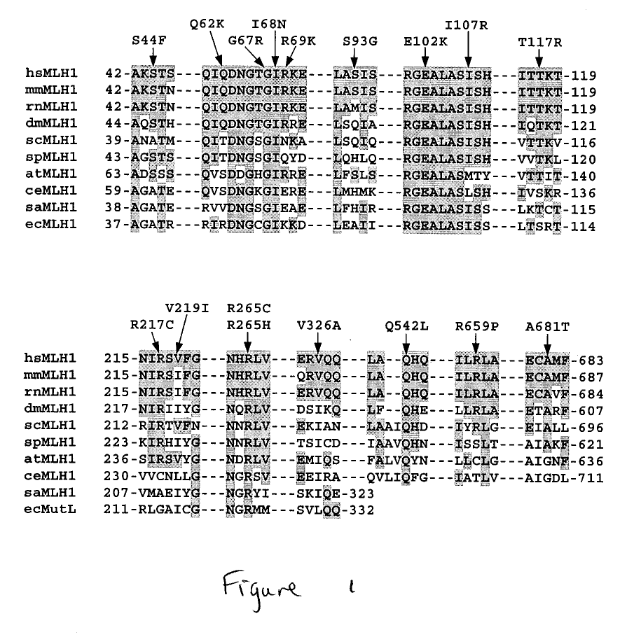

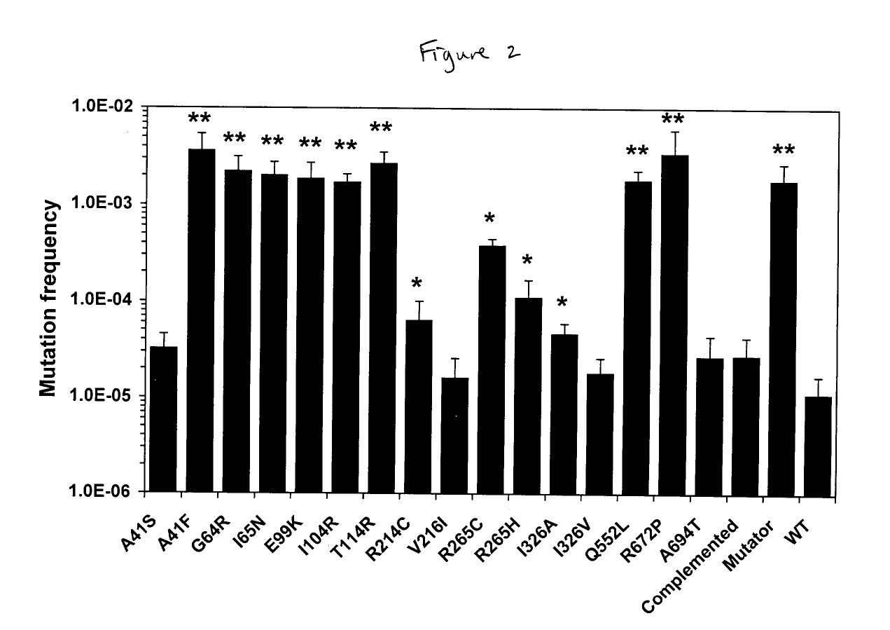

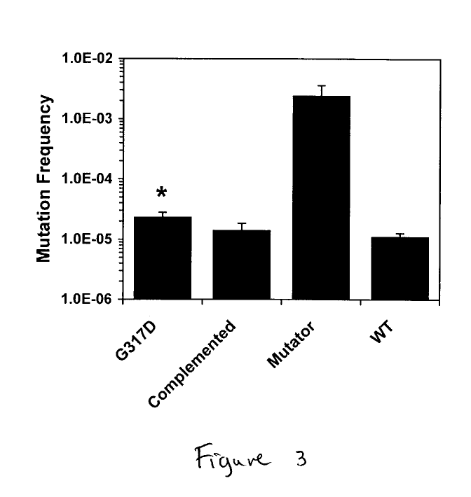

MLH1 orthologs from various organisms, including yeast and humans, were aligned to identify homologous amino acid residues (FIG. 1). These alignments, together with previously identified human missense codons, were used to target yeast residues for alterations that would mimic variants observed in the human population (Table 1). Strain YBT24 (“Mutator”, FIG. 2), exhibited a mutation frequency of 1.8×10⁻³, a level 160-fold higher than that exhibited by the wild-type parental strain YBT5-1. When the wild-type MLH1 gene (SEQ ID NO: 1) was expressed from a plasmid in YBT24 (“Complemented”, FIG. 2) the mutation frequency was reduced over 65-fold to nearly the wild-type levels. Cells expressing MLH1p with the amino acid replacements A4° F. (SEQ ID NO: 22), G64R (SEQ ID NO: 23), 165N(SEQ ID NO: 24), E99K (SEQ ID NO: 25), 1104R (SEQ ID NO: 26), Ti 14R (SEQ ID NO: 27), Q552L (SEQ ID NO: 34), and R672P (SEQ ID NO: 35) exhibited mutation frequencies of 1.7-3.6×10⁻³ (FIG. 2). These mutation frequencies represent MMR defects of 62 to 130. Statistical analyses showed these differences from the strain complemented with wild-type MLH1 to be highly significant (P<0.0001). In contrast, the mutation frequencies were not significantly different from that exhibited by the mlh1Δ deletion strain YBT24 which lacked a complementing plasmid (“Mutator”; FIG. 2). These results demonstrate that amino acid replacements A41F, G64R, 165N, E99K, 1104R, T114R, Q552L, and R672P result in complete loss of MLH1p function. These missense codons are, therefore, mutations.

Strain YBT24 expressing the A41S (SEQ ID NO: 21), V2161 (SEQ ID NO: 29), 1326V (SEQ ID NO: 33) or A694T (SEQ ID NO: 36) variants exhibited mutation frequencies of 1.0-3.2×10⁻⁵, which were not significantly different from the mutation frequency observed when strain YBT24 expressed the wild-type yeast MLH1 gene (“Complemented”; FIG. 2). The A41S and 1326V are conservative amino acid replacements. These codon changes convert the wild-type yeast residue to the amino acid in the corresponding position of the wild-type human protein. The data in FIG. 2 demonstrate that the A41S, V2161, 1326V and A694T amino acid replacements do not detectably alter MLH1p function, and therefore represent silent polymorphisms (see Description of Specific Embodiments).

Four of the codon changes in MLH1 encode proteins which appear to support intermediate efficiencies of DNA mismatch repair. The R214C (SEQ ID NO: 28), R265C (SEQ ID NO: 30), R265H (SEQ ID NO: 31) and 1326A (SEQ ID NO: 32) amino acid changes exhibit MMR defects of 2.3, 13.8, 4.0 and 1.7, respectively, which is intermediate between that of the wild-type MLH1 complemented mutant (MMR defect=1.0) and the mlh1Δ null mutant (MMR defect=65). The mutation frequencies measured with the R214C, R265C, R265H and 1326A amino acid replacements are significantly less than the mlh1Δ null mutant lacking a complementing plasmid. ANOVA/Multiple Student's T-tests statistical analyses of the mutation frequencies in each variant verses the mlhl1Δ null mutant yield P-values of less than 0.0005 in all cases. The mutation frequencies of the R214C, R265C and R265H variants are clearly different from the strain complemented with the wild-type MLH1 gene with P-values of, respectively, 0.0005, 0.0001 and 0.0001. The 1.6-fold MMR defect of the 1326A variant also appears to be significant (P=0.0279) compared to the strain expressing wild-type MLH1. These results demonstrate that the R214C, R265C, R265H and 1326A are not complete loss-of-function mutations since the MMR defect is considerably lower than that observed in the mlh1Δ null mutant. However, since the MMR defect is significantly greater than in the strain complemented with the wild-type protein, these missense variants function in DNA mismatch repair at a reduced efficiency (efficiency polymorphisms).

### EXAMPLE 6

### Functional Assay of MSH2p Variant

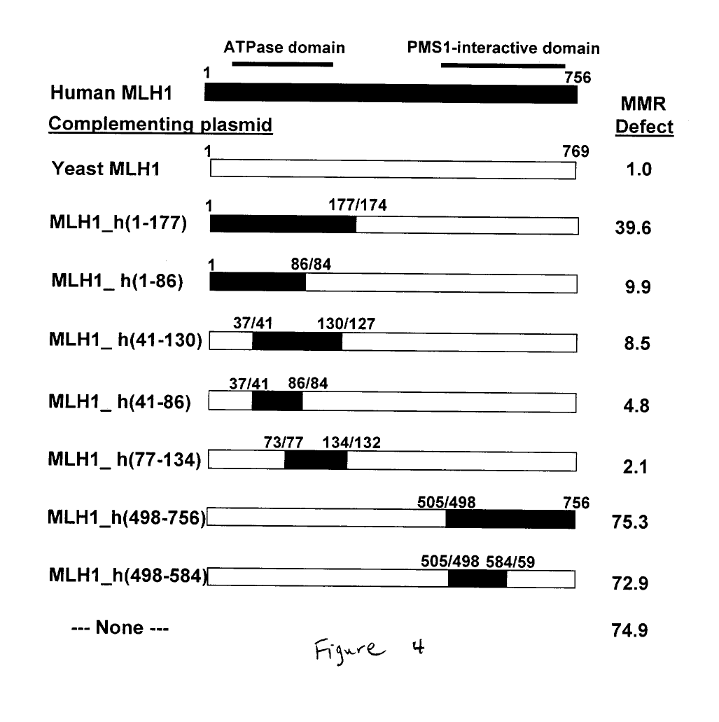

Previous work from this laboratory demonstrated that a P640L alteration in yeast MSH2p (SEQ ID NO: 124) was a loss-of-function mutation while a H658Y (SEQ ID NO: 125) alteration had no effect on protein function (Polaczek et al., 1998). In the current study, an additional amino acid replacement, G317D (SEQ ID NO: 37), in yeast MSH2p was evaluated. Mutation frequencies of strain YBT25 expressing the G317D allele was determined and compared (FIG. 3) to strain YBT25 without a complementing gene (“Mutator”) and YBT25 complemented with the wild-type MSH2 gene (“Complemented”). The strain expressing the G317D variant exhibited a low mutation frequency (2.3×10⁻⁵) that was significantly less than msh2Δ null mutant, demonstrating MMR activity of the protein variant. Cells expressing G317D, however, exhibit a MMR defect of 1.7, and this difference from YBT25 complemented with wild-type MSH2 (SEQ ID NO: 2) appears significant (P=0.0073). These results indicate that the G317D replacement encodes a functional protein of slightly reduced efficiency in MMR.

### EXAMPLE 7

### Construction of Hybrid Human-Yeast Genes Which Encode MLH1 Proteins Functional in MMR In Vivo

A substantial number of the amino acid residues in human MLH1 (SEQ ID NO: 11), which have been linked to the development of HNPCC, are not conserved in the yeast protein. As shown above (for the human S44F (SEQ ID NO: 126) and V326A (SEQ ID NO: 127) variants), alterations at non-conserved residues can be evaluated by replacing the corresponding yeast codon with that found in the native human gene. If the change is a silent polymorphism, the effect of the variant human codon can subsequently be analyzed in yeast. The utility of this approach, however, may be limited. In some cases the wild-type human amino acid may not functionally replace the yeast amino acid, or there may be instances in which the homology between the human and yeast genes is too weak to unambiguously assign the corresponding yeast codon. An alternative approach would be to construct gene fusions that encode hybrid human-yeast proteins which retain MMR function in vivo.

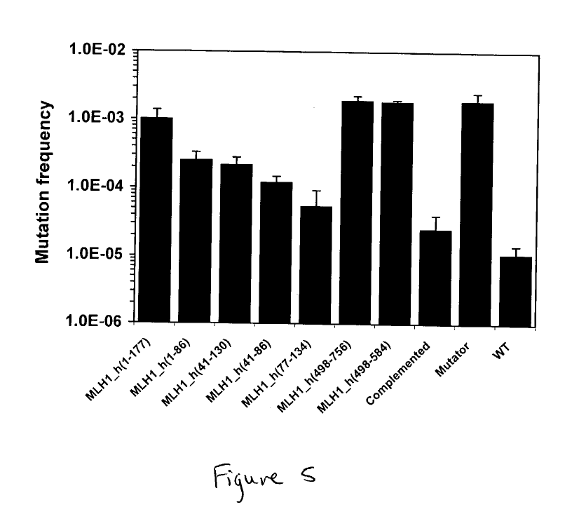

Seven gene fusions were constructed (Example 2) by replacing a portion of the yeast MLH1 gene with the homologous coding sequence from the human gene (FIG. 4A). The gene fusions were expressed from the same parental expression vector (Materials and Methods) in mlh1Δ null strain YBT24 and quantitative MMR assays were carried out to evaluate function of the hybrid human-yeast protein (FIG. 4B). Complementation of the mlh1Δ null strain was not observed with two hybrids, MLH1_h(498-756) (SEQ ID NO: 43) and MLH1_h(498-584) (SEQ ID NO: 44), that contain portions of the human C-terminal domain (FIG. 4B). In contrast, five hybrids that contained portions of the human N-terminal domain complemented strain YBT24, yielding substantial reductions in mutation frequency (FIG. 4B). The functional hybrid proteins spanned amino acids 1-177 and conferred mutation frequencies that tended to correlate with the length of the human portion of the hybrid protein. Whereas the mlh1α-null strain exhibited a MMR defect of 74.5, strains expressing hybrids MLH1_h(1-177) (SEQ ID NO: 38), MLH1_h(1-86) (SEQ ID NO: 38), MLH1_h(41-130) (SEQ ID NO: 40), and MLH1_h(41-86) (SEQ ID NO: 41) exhibited, respectively, MMR defects of 39.6, 9.9, 8.5 and 4.8. The most efficient hybrid was MLH1_h(77-134) (SEQ ID NO: 42), containing a 57 amino acid segment from the human coding sequence, which conferred an efficiency of DNA mismatch repair which is within a factor of 2 of the wild-type native yeast protein.

### EXAMPLE 8

### Evaluation of Functional Significance of Amino Acid Replacements in Human Portion of Hybrid Human-Yeast MLH1 Proteins

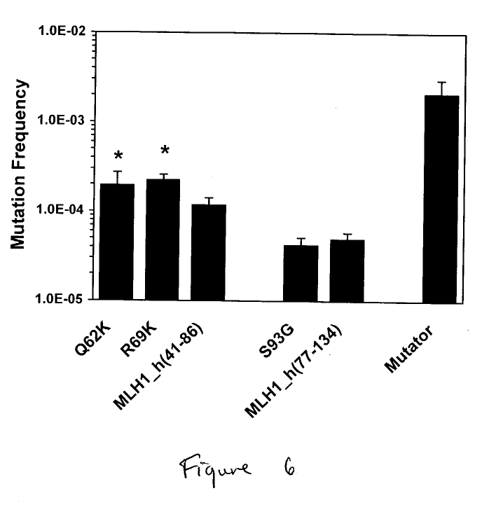

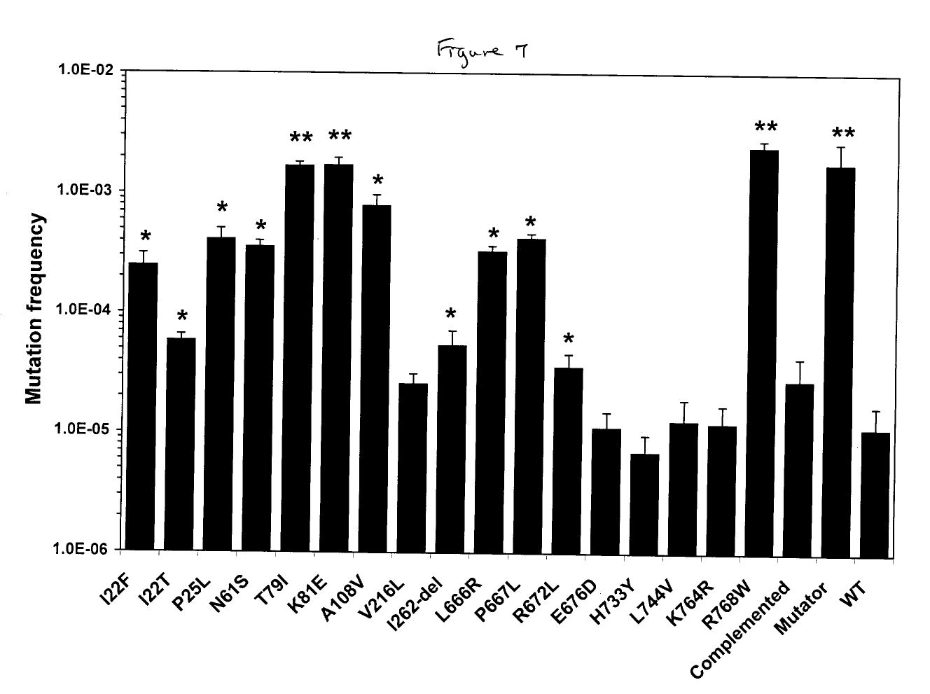

Missense codons were introduced into the human coding sequence of hybrid human-yeast genes to evaluate human amino acid replacements at residues that are not conserved in yeast. Three replacements (Q62K (SEQ ID NO: 45), R69K (SEQ ID NO: 46), S93G (SEQ ID NO: 47)) that appear in one or more of the MLH1 mutation databases were engineered by site-directed mutagenesis into an appropriate gene fusion. These genes were expressed in strain YBT24 and mutation frequencies compared to that obtained with the gene encoding the original hybrid human-yeast protein. Cells that expressed hybrid proteins with these three missense changes had mutation frequencies that were not significantly different than cells that expressed the control hybrid protein (FIG. 5). These results demonstrate that the hMLH1 S93G alteration confers no loss-of-function when present in the human portion of the hybrid protein and, thus, is a silent polymorphism while the Q62K and R69K replacements are functional at reduced efficiency and are thus efficiency polymorphisms.

### EXAMPLE 9

### Additional Reporter Genes for Functional MMR Assay

Additional reporter genes are evaluated for two purposes. First, it is possible that some amino acid replacements in MMR proteins may affect mismatch repair on certain DNA structures but not others. Although MSH2p (SEQ ID NO: 65) and MLH1p (SEQ ID NO: 15) are involved in repair of a large number of different DNA mismatch structures, it is possible that minor changes, such as amino acid replacements, in either protein may affect repair of only a subset of these structures. Thus, analyses are performed for each variant using multiple reporter genes. Second, the discovery of amino acid replacements which are not inactivating mutations but which result in decreased efficiency of DNA mismatch repair (Example 5) suggest that common polymorphisms may result in differences in efficiency of DNA mismatch repair. To improve the technology for addressing the function of these variants, additional reporter genes are evaluated for use in sensitive and quantitative DNA mismatch repair assays.

Reporter genes that have been utilized by various investigators to study DNA mismatch repair in yeast are summarized in Table 3. The type of mismatch repaired, the selection used to measure mutant frequencies and the MMR defect (mutant frequency observed in a msh2 or mlh1 mutant relative to wild type) is indicated for each reporter gene. The CAN1 gene encodes an arginine permease which is also the sole route of entry into yeast of the toxic compound canavanine. CAN1 mutants can thus be quantitated by growth in the presence of canavanine, and this selection has been shown to detect a wide range of mutations including base substitutions, frameshifts, duplications, deletions, translocations and inversions (Chen & Kolodner, 1999; Marischky et al., 1996). It is likely that utilization of the native URA3 gene (lacking a GT tract) as a reporter would reveal a similar spectrum of mutations which can be selected by resistance to 5-fluoroorotic acid (FOA). Several other reporter genes (Table V) have been described for which the MMR defect is greater than the (GT)₁₆-URA3 reporter used during Phase I. The increased sensitivity of such reporter genes may be useful for studying MLH1p or MSH2p variants which result in decreased efficiency of DNA mismatch repair (Phase I report).

Two reporter genes in addition to the (GT)₁₆-URA3 reporter (Phase I) are utilized in the functional assays (Section 4.B). The CAN1 reporter is utilized since the forward mutation assay has been shown to detect a broad spectrum of mutations. This will ensure that protein variants containing amino acid replacements which affect repair of only a subset of the possible mismatch structures are detected. The yeast strains YBT5-1, YBT24 and YBT25 are wild type for CAN1, and no further engineering will be required. A reporter gene which yields a greater MMR defect than the (GT)₁₆-URA3 reporter is also used. For example, Tran et al. (1997) constructed a series of yeast strains with homonucleotide runs of 4 to 14 A residues which produced frameshifts in the same region of the chromosomal LYS2 gene. Generally, the greater the length of the A tract, the greater was the MMR defect, reaching a maximum of 10,000 at 14 residues (Tran et al., 1997). The increased sensitivity of such a reporter gene may allow more precise functional assays of protein variants with altered efficiency of DNA mismatch repair. Srains containing such a reporter gene and which are isogenic except for the absence or presence of either a msh2A or mlh1Δ chromosomal null mutation are constructed using procedures analogous to those described in Example 3.

The reporter genes for sensitive/quantitative MMR assays are evaluated using the MLH1p R214C (SEQ ID NO: 28), R265C (SEQ ID NO: 30), R265H (SEQ ID NO: 31) and 1326A (SEQ ID NO: 32) variants (which retained MMR function but at a lower efficiency than wild type; Example 5). These amino acid replacements resulted in MMR defects measured on the (GT)₁₆-URA3 reporter gene of 2.25, 13.6, 3.9 and 1.6, respectively. In this system, the mlh1Δ null mutant (YBT24) exhibits a MMR defect of 65 (relative to YBT24 expressing a wild type MLH1p from a plasmid). A reporter gene that exhibits a greater MMR defect (e.g 10,000) allows identification and characterization of a broader spectrum of polymorphisms which result in altered efficiencies. This is evident by a greater MMR defect for the above variants, as well as a greater difference between the variants.

### EXAMPLE 10

### Secondary Assay

Secondary assays are utilized to confirm results obtained with the primary MMR functional assay (Example 4), and for generating additional information regarding the protein variant.

There is very good correlation between human clinical data on missense codons and the results of functional assays in yeast (Examples 5, 6, 8). Thus, the in vivo MMR assays in yeast appear to be predictive of protein function in humans. These assays are utilized to establish the functional significance of missense codons observed in the human hMSH2 and hMLH1 genes. There may be instances in which it seems desirable to confirm the results obtained in the primary MMR assay. In these cases, the codon change of interest is introduced into the native human gene (wild type hMSH2 (SEQ ID NO: 3) and hMLH1 (SEQ ID NO: 7)) cDNA clones, expressed in a wild type yeast strain (YBT5-1) and tested in the yeast dominant mutator assay (Clark et al., 1999; Shimodaira et al., 1998). Loss of the dominant mutator effect is consistent with a mutation, whereas no effect is consistent with the variant being a silent polymorphism.

The initial elucidation of defects in MMR as causative of HNPCC (Bronner et al., 1994; Fishel et al., 1993; Leach et al., 1993; Nicolaides et al., 1994; Papadopoulos et al., 1994) was prompted by the well documented microsatellite instability in colon cancers and the observation that DNA microsatellites are particularly unstable in yeast strains which are deficient in MMR (Strand et al., 1993). It was postulated that affected HNPCC individuals are born heterozygous for a mutation in one MMR gene and, after loss of heterozygosity due to a somatic mutation in the other allele, the resulting cell becomes a mutator. Some of these pre-cancer cells subsequently form colorectal tumors. In a subsequent study, it was demonstrated that naturally occurring mutant forms of hPMS1 function as dominant negative mutants in human cells (Nicolaides et al., 1998). That is, when expressed in a cell containing a wild type allele of the gene, the cell exhibits an elevated mutation rate. The phenomenon of dominant negative mutants of MMR proteins has also been well documented in yeast (Das Gupta & Kolodner, 2000; Drotschmann et al., 1999b; Shcherbakova & Kunkel, 1999). These observations have significance for genetic testing for HNPCC and other pre-cancers associated with defects in MMR. If a mutation is documented in one allele with the other allele remaining wild type, the cell will have a mutator phenotype if the first mutation is a dominant negative. The risk for progression to cancer is much greater for such cells, so this information will be valuable for patient counseling and management. For the amino acid replacements which are demonstrated to be mutants, it is subsequently determined whether the variant protein functions as a dominant negative mutant. The yeast expression systems (Examples 3,4) which allow complementation of chromosomal null mutants by the plasmid expressed wild type gene are used for these studies. The mutant genes are expressed in a MMR wild type strain (YBT5-1) to test for dominant negative activity.

Protein expression levels are measured by immunoblot analysis. For in vivo functional analyses, all genes are analyzed in the same yeast host containing the same reporter gene and are expressed from the same stable, single copy expression vector. The only difference in strains is the nucleotide sequence around the codon change, and the transcripitional efficiency of all genes should be equivalent. Expression of each variant protein is confirmed and quantitated relative to the wild type. Most variant proteins are expressed at levels comparable to wild type, and the functional consequences of the amino acid replacement are therefore directly attributable to effects in the process of MMR. Instances in which the variant protein is expressed less efficiently than wild type suggest that the mechanism leading to defective DNA mismatch repair for such a mutant is decreased protein stability. Epitope tagged yeast MMR proteins are also used for immonoblot analyses, but these may have subtle differences in MMR activity. Therefore, polyclonal antisera to synthetic peptides derived from the native yeast protein sequence (e.g. ref. (Drotschmann et al., 1999b)) are used for immunoblot analyses.

### EXAMPLE 11

### Additional Hybrid Human-Yeast MSH2 and MLH1 Genes

Utilization of hybrid genes encoding functional MMR proteins in which regions of yeast MSH2p (SEQ ID NO: 65) and MLH1p (SEQ ID NO: 15) are replaced by the homologous region from the human protein will further support interpretation of results obtained in yeast assays to be predictive of function in human cells. The hybrid genes will also allow assessment of the functional consequences of human amino acid replacements in regions where the homology is too weak to clearly identify the corresponding yeast residue. Finally, hybrid genes engineered such that the human segment is flanked by restriction enzyme recognition sites which are unique in the expression vector will allow random mutagenesis of the human coding region and selection of inactivating mutations.

A series of hybrid genes is constructed such that all regions of the human MSH2 (SEQ ID NO: 3) and MLH1 coding region (SEQ ID NO: 7) will be present in at least one functional hybrid protein. The technique of overlap extension PCR (Example 2) is used to construct these hybrid genes. The hybrid proteins are tested for function in vivo in strains YBT24 (MLH1 hybrids) or YBT25 (MSH2 hybrids).

Example 7 demonstrated the feasibility of constructing genes encoding functional hybrid human-yeast MMR proteins. To obtain functional hybrids with MMR efficiency equivalent to the wild type yeast protein, it appears to be necessary to limit the size of the human coding region. Attempts are made to generate functional hybrids with the largest possible human coding regions. Consideration is given, in designing such hybrids, to published data on the crystal structures (e.g. (Ban & Yang, 1998; Lamers et al., 2000; Obmolova et al., 2000)), functional domains and protein interacting regions of the MMR proteins. Additionally, restriction sites which do not alter the protein coding sequence and which are unique in the expression vector are incorporated at the ends of the human coding sequences. This will allow random mutagenesis by error prone PCR for the purpose of selecting inactivating mutations (Examples 12, 13). If it is not possible to engineer in such sites without changing the protein sequence, then nearby unique sites in the yeast coding region are used for subcloning mutagenized PCR fragments. There is the possibility that certain regions of human protein can not be incorporated into functional hybrids which are as efficient as the native yeast MLH1p (SEQ ID NO: 15). In such cases, if the hybrids exhibit significantly increased MMR activity relative to the null mutant and are only several fold less efficient than the wild type yeast protein, then they are used for retrospective and prospective analyses (Example 13). The hybrid-human yeast protein is used as the control value to which the test genes are normalized.

### EXAMPLE 12

### Positive Genetic Selections in Yeast for Mutant MMR Proteins

These selections are used for prospective analyses of mutations (Example 13).

The significance of variant MMR proteins can be assessed retrospectively (Example 13). An alternative approach is to utilize a model system to define missense codons which inactivate protein function. The data generated with such technology can be used to interpret the significance of amino acid replacements which, in the future, are observed in human proteins. Such data also provides valuable structure/function information for basic research on DNA mismatch repair. Technology is developed for selection of MSH2 and MLH1 mutants. These studies (Example 13) are readily performed with the native yeast genes to generate valuable information. However, in order to optimize application of the results to interpretation of human clinical data, the preferred embodiment uses the hybrid human-yeast genes (Examples 2, 11) and random mutagenesis is performed on the human coding sequence of the hybrid.

A qualitative spot test has been described for identifying yeast strains with increased mutation rates (Gordenin et al., 1991), and this has been applied to identify msh2 dominant negative mutants (Drotschmann et al., 1999b). In this procedure, equivalent volumes of cells from liquid cultures of different strains are spotted onto a plate which is selective for plasmids but not selective for reporter gene mutants. After incubation at 30° C., the patches of cells are replica plated to plates which are selective for mutants in the reporter gene (e.g. FOA for the (GT)₁₆-URA3 reporter; minus lysine for lys2::InsE-A₁₄; canavanine for CAN1). The amount of growth on the second plate is proportional to the mutation frequency (or MMR defect) in the strain. This qualitative test is used as one initial screen to identify MMR mutants, which are later confirmed in quantitative fluctuation tests (Example 4), The qualitative spot test also appears capable of distinguishing mutants from both wild type and variants with reduced efficiency of MMR (Drotschmann et al., 1999b). This procedure is suitable for analyzing hundreds of different yeast clones.

Technology is developed to isolate MMR mutants from larger pools of yeast (e.g. generated from libraries of mutagenized genes; Example 13). A genetic selection scheme in yeast for msh2 dominant negative mutants was recently described (Studamire et al., 1999) which employed two screens. Yeast cells contained a vector (pK5) which promoted transcription of the E. coli LacZ gene containing an out of frame (GT)₁₄ tract. This tract is much more unstable in a msh2Δ mutant; when plated on X-gal plates, the msh2Δ mutant exhibited nearly 100% blue colonies (representing frameshifts in the (GT)₁₄ tract which restores the reading frame) while a MMR wild type strain formed less than 0.5% blue colonies. The secondary assay was a qualitative spot test (above) using the CAN1 reporter gene. The msh2Δ mutant papillates to canavanine resistance at a much higher rate (50×) which is scored visually. These investigators performed random mutagenesis on the yeast MSH2 gene (SEQ ID NO: 2), and transformed the library of expression vectors containing the mutagenized MSH2 gene into yeast. Transformants were replica plated onto X-gal plates and putative MMR mutants were identified as pale-blue to blue colonies. Clones isolated in the first screen were confirmed as mutators by qualitative spot tests for canavanine resistance. From a library of 23,000 yeast transformants, 31 independent msh2 dominant negative mutants were obtained (Studamire et al., 1999). It is probable that the actual msh2 mutant frequency in the mutagenized pool was greater, since Studamire et al. (1999) selected only for dominant negative mutants by screening in a wild type yeast host.

Selections for MMR mutants are developed. The pK5 plasmid (above) is used for colorimetric mutator assays on X-gal plates. If this plasmid is not available or unsuitable for use with our strains, then a similar out of frame GT tract is engineered into a yeast expression vector which expresses the native E. coli Lac Z gene. The secondary qualitative spot test for mutators is performed with one of our other reporter genes (Example 9). The selection schemes are tested and optimized using yeast strains YBT24 (mlh1Δ null mutant) and YBT25 (msh2Δ null mutant) each with and without a plasmid expressing a wild type complementing gene. Reconstruction experiments using cultures of the wild type plasmid complemented strain seeded with various ratios of the null mutant alone are performed to test and validate the screens. Once optimized, the two step selection scheme is used to identify genes encoding missense variants that result in reduced efficiency of MMR. For these analyses, the MLH1p R265H (SEQ ID NO: 31) variant is used since it confers a mutation frequency intermediate between wild type and null mutant (Example 5). The screens allow identification of variants with reduced MMR efficiency since each screen (blue color on X-gal, spot test for mutant papillation) is qualitative. The wild type plasmid complemented and null mutants will be used as standards in optimizing the selections. A sensitive/quantitative reporter gene (Example 9) is used for the qualitative spot tests. Additional screens and selections are also developed.

### EXAMPLE 13

### Bioinformatics Applications of the Technology

There is very good correlation between human clinical data on missense codons and the results of functional assays in yeast (Example 5, 6). Thus, results in the yeast assays appear to be predictive of protein function in humans. New minor sequence alterations in hMSH2 (SEQ ID NO: 3) and hMLH1 (SEQ ID NO: 7) are continually being identified (http://www.nfdht.nl). This invention is used to determine whether amino acid replacements in hMSH2 and hMLH1 are mutations, silent polymorphisms or variants which result in reduced efficiency of MMR. The functional significance of existing and newly discovered minor gene variants is interpreted using the data which is generated as described below. This information allows accurate interpretation of genetic test results and appropriate patient counseling. Retrospective analyses.

Missense codons reported in human genes are introduced by site directed mutagenesis into the native yeast gene or, preferably, into a hybrid gene which includes the appropriate human coding region (Example 11). The efficiency of these variant proteins is determined using the three reporter genes. For mutants or altered efficiency variants, immunoblot analyses is performed to determine whether the phenotype is due to a direct effect of the altered amino acid on DNA mismatch repair or to an altered stability of the protein. It is also determined whether hybrid human-yeast proteins with amino acid replacements that are inactivating mutations function as dominant negative mutants. Prospective analyses.

The positive selections for inactivating MMR mutations (Example 12) is utilized. For each hybrid gene, the human coding region is PCR amplified from the wild type human cDNA under conditions which increase misincorporation rates. The GeneMorph PCR Mutagenesis Kit (Stratagene) is used for this purpose. The enzyme in this system incorporates 1 to 7 base substitutions per 1000 bp, and fewer than 2% of the in vitro alterations are insertions or deletions. Thus, for a human coding region of 50 codons (150 bp), using the most error prone conditions should yield approximately one base substitution per DNA molecule. The PCR products are ligated into appropriately restricted and gel purified parental expression vector and transformed into E. coli. A library of hybrid genes (>110,000 independent clones) is transformed into the appropriate yeast strain (YBT25 for MSH2 hybrids; YBT24 for MLH1 hybrids; each with appropriate reporter genes) selecting for histidine prototrophs (marker on the expression vector; Example 1). Subsequently, the library of yeast is subjected to the two step screen for mutator phenotypes (Example 12). For each mutator identified, immunoblot analysis is performed to determine the size of the MMR protein. For yeast mutators expressing full length MMR protein, the expression vector is shuttled into E. coli by standard methods. The human coding region is subjected to DNA sequence analysis to determine the alteration which gives rise to the defect in MMR. (Note that all regions of the plasmid are derived from an expression vector that complements the null mutation with the exception of the PCR generated human coding region.) Yeast strains expressing the mutant MMR protein are also tested in quantitative MMR fluctuation tests to determine whether the protein variant is a loss of function mutant (MMR defect equivalent to the null mutant) or an MMR variant of reduced efficiency.

### EXAMPLE 14

### Construction of Additional Hybrid Human-Yeast MLH1 Genes.

Hybrid MLH1 h(77-177) (SEQ ID NO: 128) was constructed using a two-piece overlap extension. A 284-bp fragment of the human MLH1 coding region was amplified by PCR from hMLH1 cDNA clone ATCC#217884 using primers E466-2 and D650-4 (Example 2). A 1813-bp fragment of the yeast MLH1 C-terminal coding sequence and 3′ untranslated region was amplified by PCR from S. cerevisiae strain S288C genomic DNA using primers D173-5 and T941-6 (Example 2). The two fragments were diluted and combined in approximately equimolar amounts and subjected to overlap extension PCR using primers E466-2 and T941-6. All PCR amplifications were carried out using Pfu DNA polymerase (Stratagene, La Jolla) and employed conditions recommended by the manufacturer. The overlap extension PCR fragment was digested with AatII and XhoI and ligated into AatII-XhoI digested pMLH1. DNA sequencing was carried out to confirm the sequence of the hybrid gene.

A plasmid containing MLH1_h(77-177) (SEQ ID NO: 128) was introduced into YBT24;pSH91 as described in Example 3 and MMR assays were carried out as described in Example 4. The results demonstrated that the hybrid complemented the MLH1 deficiency. The mean mutation frequency of yeast strain that carried this hybrid was 5.9×10⁻⁵, a level approximately the same as observed with the hybrid MLH1_h(77-134) (SEQ ID NO: 42) and significantly less than the null mutant (1.6×10⁻³). These results indicate that the hybrid MLH1_h(77-177) (SEQ ID NO: 128) is functional in MMR and exhibits a mutation defect only 2.1-fold higher than yeast complemented with the wild-type MLH1 gene.

### EXAMPLE 15

### Construction of a Hybrid Human-Yeast MSH2 Gene.

Hybrid gene MSH2_h(621-832) (SEQ ID NO: 129) was constructed using a three-piece overlap extension PCR. A 681-bp fragment of the human MSH2 coding sequence was amplified by PCR from hMSH2 cDNA clone ATCC#788421 using primers SEQ ID NO: 130 and SEQ ID NO: 131. A 1168-bp fragment encompassing the central portion of the yeast MSH2 coding sequence was amplified from S. cerevisiae strain S288C genomic DNA using primers SEQ ID NO: 132 and SEQ ID NO: 133. A 344-bp fragment encompassing the C-terminal coding region and 3′ untranslated region of yeast MSH2 was amplified from S. cerevisiae strain S288C genomic DNA using primers SEQ ID NO: 134 and SEQ ID NO: 135. The three fragments were diluted, mixed in approximately equimolar amounts and subjected to overlap extension PCR using primers SEQ ID NO: 132 and SEQ ID NO: 135. All PCR amplifications were carried out using Pfu DNA polymerase (Stratagene, La Jolla) or PfuTurbo (Stratagene) and employed conditions recommended by the manufacturer. The overlap extension PCR fragment was digested with SphI and XhoI and ligated into SphI-XhoI digested pMETc/MSH2. DNA sequencing is carried out to confirm the sequence of the hybrid gene. Function of the hybrid protein encoded by gene in DNA mismatch repair is determined according to the method in Example 4 and compared to the mlh1 null strain and the mlh1 null strain complemented with a plasmid expressed wild type MLH1 gene.

### EXAMPLE 16

### Additional Functional Assays of Mlh1p Variants Containing Amino Acid Replacements

During the process of screening for mutations in MMR genes it is common to find missense mutations and small in-frame deletions in the coding region of MMR genes. The functional significance of these alterations in MMR is uncertain and hence these changes have been termed variants of “uncertain significance” and “questionable pathogenicity”. A current review of the MLH1 mutation databases listed in Example 1, single nucleotide polymorphism (SNP) databases and the literature (Cunningham et al., 2001; Jakubowska et al., 2001; Syngal et al., 1999; Terdiman et al., 2001) has revealed more than 80 missense alterations and in-frame deletions that could be characterized as variants of uncertain significance. Note that numbers refer to the codon position (amino acid reside) in the full-length open reading frame with position 1 being the start codon (methionine).

To determine the function of Mlh1p variants site-directed mutations were introduced into the yeast MLH1 gene as described in Example 1. Table 4 lists amino acid substitutions observed in human MMR genes and the homologous codon change introduced into the yeast orthologue. Plasmid pMLH1 was used as the template for the derivation of MLH1 variants 122F (SEQ ID NO: 48), I22T (SEQ ID NO: 49), P25L (SEQ ID NO: 50), N61S (SEQ ID NO: 51), T791 (SEQ ID NO: 52), K81E (SEQ ID NO: 53), A108V (SEQ ID NO: 54), V216L (SEQ ID NO: 55), I262-del (SEQ ID NO: 56), L666R (SEQ ID NO: 57), P667L (SEQ ID NO: 58), R672L (SEQ ID NO: 59), E676D (SEQ ID NO: 60), H733Y (SEQ ID NO: 61), L744V (SEQ ID NO: 62), K764R (SEQ ID NO: 63), and R768W (SEQ ID NO: 64). Table 4 also lists the DNA sequences and restriction site alterations introduced by the oligonucleotide used for site-directed mutagenesis of the yeast gene. The variant MLH1 genes were introduced into yeast strain YBT24;pSH91 and functionally tested in the standardized MMR assay as described in Examples 1, 4 and 5 except that total cell number was calculated using OD₅₉₅ measurements from an aliquot of the yeast culture and a conversion factor of 1.0 OD₅₉₅=1.1×10⁷ viable cells was applied. At least 3 independent mutant clones were tested for function in yeast with identical results. At least one clone was sequenced to confirm the appropriate codon change. The data presented are derived from four replicate cultures of a single mutant clone that had been confirmed by DNA sequencing. Statistical comparisons were done using unpaired, two-tailed t-tests (Excel version 95, Microsoft) and the null hypothesis was rejected for P-values≦0.05.

Five of the codon changes in MLH1 (V216L (SEQ ID NO: 55), E676D (SEQ ID NO: 68), H733Y (SEQ ID NO: 61), L744V (SEQ ID NO: 62), and K764R (SEQ ID NO: 63)) encode proteins that complemented the MLH1-deficient strain YBT24. The mean mutation frequencies as determined using the standardized MMR assay ranged from 6.9×10⁻⁶ to 2.6×10⁻⁵ (FIG. 7). A statistical analysis (t-tests) showed that these values were not significantly different from the control strain complemented with the wild-type MLH1 gene (“Complemented”, FIG. 7). The results indicate that the Mlh1p alterations V216L, E676D, H733Y, L744V, and K764R are silent polymorphisms. Pang et al. (Pang et al., 1997) previously observed that the K764R alteration in Mlh1p does not impair reversion frequency of a different reporter gene, the hom3-10 allele, in yeast.

Three of the codon changes in MLH1 (T791 (SEQ ID NO: 52), K81E (SEQ ID NO: 53) and R768W (SEQ ID NO: 64) strongly impaired MMR activity. The mean mutation frequencies ranged from 1.3×10⁻³ to 2.5×10⁻³; values that were not greatly different from the MLH1-deficient strain (“Mutator”, FIG. 7). The results indicate that the Mlh1p alterations T791, K81E and R768W confer complete or near complete loss of MMR function and thus these alterations are considered to be mutations.

Nine of the codon changes in MLH1 (122F (SEQ ID NO: 48), I22T (SEQ ID NO: 49), P25L (SEQ ID NO: 50), N61S (SEQ ID NO: 51), A108V (SEQ ID NO: 54), 1262-del (SEQ ID NO: 56), L666R (SEQ ID NO: 57), P667L (SEQ ID NO: 58), R672L (SEQ ID NO: 59) encode proteins with intermediate efficiencies of MMR. The mean mutation frequencies ranged from 3.6×10⁻⁵ to 7.8×10⁻⁴; values that were signficantly less than the MLH1-deficient strain (“Mutator”, FIG. 7) but significantly more than the MLH1p complemented strain (“Complemented”, FIG. 7). The results indicate that the Mlh1p alterations I22F, I22T, P25L, N61S, A108V, 1262-del, L666R, P667L and R672L confer partial function in MMR and thus are considered to be efficiency polymorphisms. An Mlh1 protein with the P25L alteration was previously observed to have intermediate levels of MMR activity using different reporter genes (Shcherbakova & Kunkel, 1999).

### EXAMPLE 17

### Additional Functional Assays of Msh2p Variants Containing Amino Acid Replacements

Msh2p variant R542P (SEQ ID NO: 172) was constructed using two-piece overlap extension PCR. An 874-bp upstream fragment of MSH2 was amplified from S. cerevisiae strain S288C genomic DNA using primer SEQ ID NO: 132 (Example 15) and the MSH2 R542P (SEQ ID NO: 171) antisense primer (Table 4). A 393-bp downstream fragment of MSH2 was amplified from S. cerevisiae strain S288C genomic DNA using the MSH2 R542P (SEQ ID NO: 170) sense primer (Table 4) and SEQ ID NO: 173. Both fragments were amplified with Pfu polymerase (Stratagene). The upstream and downstream fragments were diluted and mixed in approximately equimolar amounts and subjected to overlap extension PCR using Taq polymerase and primers SEQ ID NO: 132 and SEQ ID NO: 173. The overlap extension product was digested with BglII and NcoI and subcloned into BglII-NcoI digested pBluescript-yMSH2. Individual clones were identified which contained the MSH2 R542P variant and full-length BamHI-XhoI MSH2 fragments were cloned into BamHI-XhoI digested expression vector pMETc. This construct identical to the pMETc/MSH2 expression vector except that it contains the R542P alteration. The alteration and yeast sequences, including the entire BglII-NcoI fragment that was amplified by overlap extension, were confirmed by DNA sequence analysis.

The MSH2 R542P expression construct was introduced into YBT25;pSH91 as described in Example 3 and MMR assays were carried out as described in Example 4. The mutation frequency of the representative yeast strain was 1.33×10⁻⁴, a level which was intermediate between the MSH2p-deficient strain, YBT25; pSH91, pMETc (2.39×10⁻³), and the MSH2-complemented strain, YBT25; pSH91, pMETc/MSH2 (1.57×10⁻⁵). The results indicate that the Msh2p alteration R542P confers partial function in MMR and is therefore an efficiency polymorphism. The results differ from a previous report which suggested that the MSH2 R542P alteration strongly inactivated MMR (Drotschmann et al., 1999a).

### EXAMPLE 18

### Prospective Screen for MMR Gene Variants that Cause Loss of MMR Function

New methodology was developed to identify novel MMR gene variants (not previously observed) that impair MMR activity. The screen utilizes hybrid human-yeast genes to allow the identification of critical human codons within the human portion of the hybrid gene. As described in detail below the methodology incorporated (i) generation in yeast of a library of MMR gene variants, (ii) screening of the yeast library for strains deficient in MMR, (iii) identification of the causative mutation in the MMR gene and (iv) validation of the causative mutation in standardized MMR assays. The methodology has been used to identify 39 novel MLH1 amino acid replacements that impair MMR function (see Results below).

Method (i): The generation in yeast of a library of MMR gene variants. In vivo gap repair cloning in yeast (Ishioka et al., 1993; Scharer & Iggo, 1992) was used to create a library of yeast strains that contain nucleotide alterations in a portion of hybrid human-yeast MLH1 genes. The gap repair assay in yeast is a highly effective technique to clone DNA fragments and is based on efficient homologous recombination mechanisms in yeast when the introduced DNAs contain regions of overlapping homology. The vector for in vivo gap repair was ClaI-AatII digested pMLH1, which deletes codons 38-83 of yeast MLH1. The mutant 401-bp fragments used for gap repair were generated by error-prone PCR using XhoI-linearized pMLH1_h(41-86) as a template for amplification. The upstream and downstream primers used for amplification of the MLH1 fragment were SEQ ID NO: 174 and SEQ ID NO: 175, respectively. In preliminary experiments the upstream primer SEQ ID NO: 176 was used to produce a fragment of 475-bp. Several different PCR conditions were used to generate mutant MLH1 gene fragments. PCR mixes utilized either Taq DNA polymerase (Promega) or Mutazyme DNA polymerase (Stratagene) and the reaction buffer, nucleotides, primers and enzyme concentrations recommended by the manufacturer. Conditions of high and low fidelity were manipulated by varying the MgCl₂ concentration (1.5-2.5 mM) in reactions employing Taq DNA polymerase and the amount of input DNA (3-74 ng) in reactions employing Mutazyme polymerase. The protocol for PCR temperature cycling was as follows: 94° C. for 2 min; 33 cycles of 94° C. for 36 sec, 55° C. for 1 min, 72° C. for 2 min; and 72° C. for 10 min. The resulting PCR fragments were purified with Wizard PCR preps (Promega).

For gap repair in yeast approximately 0.5 μg purified PCR product was combined with 0.4 μg gapped vector DNA and introduced into yeast strain YBT24; pSH91 by lithium acetate-polyethelene glycol transformation as described in Example 3. Yeast cells in which fragment and vector recombined to produce a circular replicating plasmid were converted to histidine prototrophy due to to presense of the HIS3 marker gene present on the pMLH1 vector and were selected by growth on SD plates supplemented with adenine and lysine. Typically 300-700 colonies were obtained on these plates while plates that received yeast cells transformed with vector or fragment alone exhibited very few (<5) colonies. The individual colonies that grew as a result of the in vivo gap repair technique are expected to contain products of the homologous recombination between genetically distinct mutant MLH1 gene fragments and the gapped vector DNA and therefore constitute a library of hybrid human-yeast MLH1 genes.

In addition to the generation of libraries derived from the hybrid MLH1_h(41-86) (SEQ ID NO: 41), libraries derived from the hybrid MLH1 h(77-134) (SEQ ID NO: 42) have been generated by gap repair cloning in yeast. The same methodology as described above was used except that XhoI-linearized pMLH1_h(77-134) was used as the template for PCR. Identical PCR conditions were employed and transformation of yeast utilized the same ClaI-AaII digested pMLH1 vector DNA.

Method (ii): Screening of the yeast library for strains deficient in MMR. The proficiency of MMR of individual yeast clones from the gap repair transformation was determined using a series of qualitative MMR assays which measure genetic instability of reporter genes. The first screen, based on the in vivo MMR assay described in Example 4, was adapted for high through-put using small culture volumes and a qualititive spot test as follows: Individual yeast clones were grown overnight in glass test tubes at 30° C. in 3 ml SD medium supplemented with adenine and lysine with vigorous shaking (Day 1 culture). The next day 120 μl was subinoculated into 3 ml SD medium supplemented with adenine, lysine and uracil and again grown overnight (Day 2 culture). The next day 4 μl of the saturated Day 2 culture was spotted in duplicate on SD plates supplemented with adenine, lysine, uracil and containing 1 mg/ml FOA. The plates were incubated at 30° C. for two to three days and then scored by counting the number of FOA-resistant colonies on each spot. Strains that exhibited few colonies (typically <15) were scored as having low levels of genetic instability (i.e. normal in MMR) and were discarded. Strains that exhibited many colonies (typically >15) were scored as having high levels of genetic instability (i.e. deficient in MMR) and were selected for further analysis. These strains were arrayed on a master plate by applying 25 μl of the Day 1 cultures to SD plates supplemented with adenine and lysine and grown for two days. The secondary screen used to examine the proficiency of MMR was based on spontaneous mutation of the CAN1 gene as described in Example 9. A loopful of each each strain was taken from the master plate and patched onto SD plates supplemented with adenine, lysine and containing 60 μg/ml canavanine. Plates were incubated three to four days at 30° C. and scored by counting the number of canavanine-resistant colonies that grew. Strains that exhibited few colonies (typically<15) were scored as having low levels of genetic instability (i.e. normal in MMR) and were discarded. Strains that exhibited many colonies (typically>15) were scored as having high levels of genetic instability (i.e. deficient in MMR). These clones, which exhibited high levels of genetic instability in both screens, were selected for further analysis.

Method (iii): Identification of the causitive mutation in the MMR gene. Total yeast DNA was prepared from yeast clones of interest using the glass-bead method (Hoffman & Winston, 1987) modified as follows: Yeast clones were grown to saturation in 20 ml SD medium supplemented with adenine and lysine. Yeast cells from a 7 ml aliquot of this culture were collected by centrifugation (2500 rpm, 10 min), washed in H₂O, transferred to an eppendorf tube, centrifuged (10,000 rpm, 5 see) and resuspended in 200 μl cell lysis buffer containing 0.1 M NaCl, 10 mM Tris-HCl, 1 mM EDTA, 2% (vol/vol) Triton X-100, 1% (wt/vol) SDS and 10 μg/ml RNase A (Qiagen). To disrupt the cells an equal volume of acid-washed glass beads (#G-8772, Sigma) was added and the samples were vortexed for 3 min. Samples were incubated 10 min at room temperature, phenol-chloroform extracted, precipitated in ethanol and the resulting DNA preparation was resuspended in 50 μl H₂O. Before transformation of bacteria with the DNA preparation, a 15 μl aliqout of each sample was digested with BamHI, which restricts only the reporter plasmid pSH91, and thus creates a strong bias toward transformation with the mutant MLH1 plasmids. Three microliters of the BamHI-digested DNA was introduced into E. coli strain DH5α by electroporation using an electroporator (BTX model #ECM399, Gentronix, Inc.) and following the manufactorures instructions. Colonies containing the mutant MLH1 plamids were selected by growth on LB plates containing 50 μg/ml ampicillin. Plasmid DNA was purified using the Wizard Plus SV Minipreps kit (Promega) and sequenced as described in Example 1 over the portion of the MLH1 gene corresponding to the entire mutagenized PCR fragment. Sequencing was carried out in both the forward and reverse direction using primers SEQ ID NO: 177 and SEQ ID NO: 175 (above), respectively.

Method (iv): Validation of the causative mutation in standardized MMR assays. Mutant MLH1 plasmids were selected for further study if they were found to contain a single codon change in the MLH1 gene, since it appeared that the codon change in each plasmid was responsible for the impaired MMR phenotype. However it was also formally possible that the mutator phenotype might also have been a result of spontaneous mutations in the reporter gene and/or other endogenous genes in that particular yeast clone. To determine capability for MMR of the gene encoded by the isolated plasmid, the recovered plasmids were reintroduced into yeast strain YBT24;pSH91 by transformation as described in Example 3. Colonies were selected by growth on SD plates containing adenine and lysine and two transformants with each revovered plasmid were tested in quantitative in vivo MMR assays as described in Example 4. Strains that exhibited a high mutation frequency as compared to the appropriate control strain [YBT24;pSH91, pMLH1_h(41-86) or YBT24;pSH91, pMLH1_h(77-134)] were scored as containing plasmids with a codon change that impaired MMR. It should be noted that the mutant and control strains were genetically identical except for the single codon change present in the MLH1 gene.

Results: Approximately 600 transformants [200 MLH1_h(41-86) and 400 MLH1_h(77-134)] were functionally screened following the gap repair cloning method in yeast. Aproximately 130 (22%) of these exhibited a strong mutator phenotype when analyzed by Method (ii), and were sequenced to determine the genetic alteration in the MLH1 gene. A variety of alterations were observed including nucleotide insertions, deletions, nonsense and missense mutations. The ratio and spectra of the mutations was very similar to those previously reported for Taq and Mutazyme DNA polymerase (Stratagene GeneMorph PCR mutagenesis kit product literature and references therein). Forty-eight (37%) of the mutant MLH1 molecules contained a single missense alteration and 39 of these were novel alterations that impaired MMR activity when validated by Method (iv) [The remainder either were duplicate alterations (n=6) or exhibited substantial MMR activity when reintroduced into yeast (n=3)]. The 39 novel MMR gene inactivating alterations have been listed in Table 5. Of note is the fact that 3 alterations are identical to changes previously observed in HNPCC patients (hMLH1 G67E, C77R and K84E) and 4 alterations occur at the same codon where different substitutions were previously reported in HNPCC (hMLH1 S44V, G67V, C77G, K84R).

### Example 19

### Functional Analysis of MLH1 Proteins having Missense Alterations at Human Codon S44.

To determine how phenotypic variability relates to genetic variablity at a single amino acid a spectrum of codon alterations were created in the human-yeast hybrid MLH1_h(41-86) (SEQ ID NO: 41) at the amino acid residue corresponding to human codon 44 (S44). An oligonucleotide pool with the sequence SEQ ID NO: 178, where “N” represents any of the four nucleotides A, C, G or T, was synthesized by and purchased from Bio-Synthesis Inc. (Lewisville, TX). Ideally, the random incorporation of nucleotides at this triplet, creates a collection of oligonucleotides containing all 64 possible codon (encoding all 20 possible amino acids plus 3 termination codons) alterations at this position. Oligonucleotide pool SEQ ID NO: 178, in combination with SEQ ID NO: 114 (Example 2), was then used to amplify a portion of the hMLH1 gene using hMLH1 cDNA clone ATCC#217884 as a template. Amplification utilized Pfu DNA polymerase (Stratagene) according to the manufacturer's instructions and cycling conditions were as follows: 94° C. for 2 min; 30 cycles of 94° C. for 36 sec, 55° C. for 1 min, 72° C. for 2 min 30 sec; and 72° C. for 10 min. The resulting 125-bp fragment was digested with C/al and AatII and ligated into pMLH1 replacing a portion of the native MLH1 gene (SEQ ID NO: 1). Cloning generates a pool of molecules identical to pMLH1_h(41-86) (see Example 2) except for the randomized codon at hMLH1 codon 44. Transformation into E. coli DH5α generated a collection of colonies that each contain a unique pMLH1_h(41-86) molecule. Plasmid DNA from individual colonies was purified using Wizard Plus SV Minpreps (Promega) and then analyzed by DNA sequencing (Example 1) to confirm the sequence of the amplified region and, importantly, to determine the codon present at hMLH1position 44. Plasmids that contained a novel change at position 44 were transformed into YB24;pSH91 (Example 3) and two independent colonies from each yeast transformation were tested using the standardized quantitative MMR assay (Example 4).

Codons corresponding to 14 of the 20 possible amino acids have been identified and functionally characterized. One alteration was a silent change (S44S) (SEQ ID NO: 179), one alteration (S44A) (SEQ ID NO: 180) encoded a protein with wild-type levels of MMR activity and 12 alterations confered complete loss of MMR function. As detailed in Table 6, serine (S) and alanine (A) at amino acid 44 of hMLH1p resulted in proteins with normal MMR activity, and the substitutions arginine (R) (SEQ ID NO: 181), cysteine (C) (SEQ ID NO: 182), glutamine (O) (SEQ ID NO: 183), histidine (H) (SEQ ID NO: 184), isoleucine (I) (SEQ ID NO: 185), leucine (L) (SEQ ID NO: 186), phenylalanine (F) (SEQ ID NO: 187), proline (P) (SEQ ID NO: 188), threonine (T) (SEQ ID NO: 189), tryptophan (W) (SEQ ID NO: 190), tyrosine (Y) (SEQ ID NO: 191), and valine (V) (SEQ ID NO: 192) resulted in proteins with impaired MMR activity. In confirmation of these findings it should be noted that alanine (A) is the normal amino acid at the corresponding position in the yeast protein (MLH1p) and an alanine (A) to serine (S) alteration in MLH1p was previously shown to results in a protein with normal MMR activity (Example 5). Furthermore, a serine (S) to phenylalanine (F) alteration has been reported in families with HNPCC (Bronner et al., 1994; Hackman et al., 1997; Tannergard et al., 1995), and the introduction of phenylalanine (F) at the corresponding position in yeast MLH1p (S41 F) abolished MMR activity (Example 5 and (Pang et al., 1997)). The results indicate that the majority of missense alterations at hMLH1 codon 44 impair MMR activity. In addition to showing functional variability due to changes at a single codon, the results of these experiments provide additional prospective information about missense alterations that impair MMR and thus, may have a role in the development of human cancers.

TABLE 1
Human MLHJ and MSH2 variants examined in this study and oligonucleotides used for
making site-directed mutations

| | | Equivalent | | | | Restriction |
|---|---|---|---|---|---|---|
| Variation in | | substitution | Oligonucleotide sequence^{a} and SEQ ID# | | | site |
| human MMR gene | | in yeast | [(S), sense and (A), anti-sense strand] | | | alteration^{b} |
| MLHI | S44F | A41F | 5′-CCATCGATGCGAACTTTACAATGATTGATATTC-3′ | (S) | (SEQ ID 69) | −BsaMI |
| | | | 5′-GAATATCAATCATTGTAAAGTTCGCATCGATGG-′3 | (A) | (SEQ ID 70) | |
| | — | A41S | 5′-CCATCGATGCGAATTCTACAATGATTGATATTC-3′ | (S) | (SEQ ID 71) | +EcoRI |
| | | | 5′-GAATATCAATCATTGTAGAATTCGCATCGATGG-′3 | (A) | (SEQ ID 72) | |
| | Q62K | — | 5′-GAAGTTGATTCAGATTAAAGACAATGGCACCG-3′ | (S) | (SEQ ID 73) | −BstXI |
| | | | 5′-CGGTGCCATTGTCTTTAATCTGAATCAACTTC-′3 | (A) | (SEQ ID 74) | |
| | G67R | G64R | 5′-GATAACGGATCGCGAATTAATAAAGCAGACCTGCC-3′ | (S) | (SEQ ID 75) | +NruI |
| | | | 5′-GGCAGGTCTGCTTTATTAATTCGCGATCCGTTATC-′3 | (A) | (SEQ ID 76) | |
| | 168N | 165N | 5′-GATAACGGATCTGGAAATAATAAAGCAGAC-3′ | (S) | (SEQ ID 77) | −VspI |
| | | | 5′-GTCTGCTTTATTATTTCCAGATCCGTTATC-′3 | (A) | (SEQ ID 78) | |
| | R69K | — | 5′-CAATGGCACCGGTATCAAGAAAGAAGATCTGG-3′ | (S) | (SEQ ID 79) | +AgeI |
| | | | 5′-CCAGATCTTCTTTCTTGATACCGGTGCCATTG-′3 | (A) | (SEQ ID 80) | |
| | S93G | — | 5′-CTTTGAGGATTTAGCCGGTATTTCTACCTATG-3′ | (S) | (SEQ ID 81) | +BsrFI |
| | | | 5′-CATAGGTAGAAATACCGGCTAAATCCTCAAAG-′3 | (A) | (SEQ ID 82) | |
| | E102K | E99K | 5′-GGATTCCGAGGAAAGGCTTTAGCCAGTATCTC-3′ | (S) | (SEQ ID 83) | −HindIII |
| | | | 5′-GAGATACTGGCTAAAGCCTTTCCTCGGAATCC-′3 | (A) | (SEQ ID 84) | |
| | I107R | I104R | 5′-GAAGCTTTAGCAAGTAGATCTCATGTGGCAAGAG-3′ | (S) | (SEQ ID 85) | +BglII |
| | | | 5′-GAAGCTTTAGCAAGTAGATCTCATGTGGCAAGAG-′3 | (A) | (SEQ ID 86) | |
| | T117R | T114R | 5′-GAGTCACAGTAACGCGTAAAGTTAAAGAAGAC-3′ | (S) | (SEQ ID 87) | +MlnI |
| | | | 5′-GTCTTCTTTAACTTTACGCGTTACTGTGACTC-′3 | (A) | (SEQ ID 88) | |
| | R217C | R214C | 5′-CCAGGATAGGATTTGTACAGTGTTCAATAAATC-3′ | (S) | (SEQ ID 89) | +BsrGI |
| | | | 5′-GATTTATTGAACACTGTACAAATCCTATCCTGG-′3 | (A) | (SEQ ID 90) | |
| | V219I | V216I | 5′-GATAGGATTAGGACAATATTCAATAAATCTGTG-3′ | (S) | (SEQ ID 91) | +SspI |
| | | | 5′-CACAGATTTATTGAATATTGTCCTAATCCTATC-′3 | (A) | (SEQ ID 92) | |
| | R265C | R265C | 5′-TTTTTTTCATTAATAATTGTCTAGTGACATGTG-3′ | (S) | (SEQ ID 93) | −SpeI |
| | | | 5′-CACATGTCACTAGACAATTATTAATGAAAAAAA-′3 | (A) | (SEQ ID 94) | |
| | R265H | R265H | 5′-TTTTTTTCATTAATAATCACCTAGTGACATGTG-3′ | (S) | (SEQ ID 95) | −SpeI |
| | | | 5′-CACATGTCACTAGGTGATTATTAATGAAAAAAA-′3 | (A) | (SEQ ID 96) | |
| | V326A | I326A | 5′-GAGATCATAGAGAAAGCGGCCAATCAATTGC-3′ | (S) | (SEQ ID 97) | +EaeI |
| | | | 5′-GCAATTGATTGGCCGCTTTCTCTATGATCTC-′3 | (A) | (SEQ ID 98) | |
| | — | I326V | 5′-GAGATCATAGAGAAAGTCGCGAATCAATTGC-3′ | (S) | (SEQ ID 99) | +NruI |
| | | | 5′-GCAATTGATTCGCGACTTTCTCTATGATCTC-′3 | (A) | (SEQ ID 100) | |
| | Q542L | Q552L | 5′-GATTAGCCGCTATTCTTCATGACTTAAAGC-3′ | (S) | (SEQ ID 101) | +BspHI |
| | | | 5′-GCTTTAAGTCATGAAGAATAGCGGCTAATC-′3 | (A) | (SEQ ID 102) | |
| | R659P | R672P | 5′-CCATTTTTTATATATCCCTTAGGTAAAGAAGTTG-3′ | (S) | (SEQ ID 103) | +Bsu36I |
| | | | 5′-CAACTTCTTTACCTAAGGGATATATAAAAAATGG-′3 | (A) | (SEQ ID 104) | |
| | A681T | A694T | 5′-GTATTTTAAGAGAGATTACATTGCTCTATATACCTG-3′ | (S) | (SEQ ID 105) | +BsrDI |
| | | | 5′-CAGGTATATAGAGCAATGTAATCTCTCTTAAAATAC-′3 | (A) | (SEQ ID 106) | |
| MSH2 | G322D | G317D | 5′-CAAAATCCATTCGATAGCAACAATTTAGC-3′ | (S) | (SEQ ID 107) | None^{c} |
| | | | 5′-GCTAAATTGTTGCTATCGAATGGATTTTG-′3 | (A) | (SEQ ID 108) | |

^{a}Both sense and antisense oligonucleotides were employed for site-directed mutations in yeast and human MMR genes as described in the Materials and Methods section.
^{b}Restriction site alterations are silent, except for the indicated amino acid substitutions. +, restriction site additon; −, restriction site loss.
^{c}Alteration screened by DNA sequencing.

TABLE 2
Comparison of functional and clinical data concerning MMR gene variants

| Variation | | Equivalent | Patient satisfies | Mutation | | |
|---|---|---|---|---|---|---|
| human MMR | | subsitution | ICG-criteria^{a} | segregates | Functional classification^{a} | |
| gene | | in yeast | for HNPCC | with disease | (Results) | Other functional data |
| MLH1 | S44F | A41F | Yes | Yes | Mutant | Mutant (Pang et al., 1997) |
| | — | A41S | — | — | Polymorphism | Polymorphism (Pang et al., 1997) |
| | Q62K | — | Yes | ? | Δ Efficiency | — |
| | G67R | G64R | Yes | No | Mutant | Mutant (Pang et al., 1997) |
| | I68N | I65N | Yes | ? | Mutant | Mutant (Pang et al., 1997) |
| | R69K | — | No | ? | Δ Efficiency | — |
| | S93G | — | No | ? | Polymorphism | — |
| | E102K | E99K | ? | ? | Mutant | — |
| | I107R | I104R | Yes | ? | Mutant | — |
| | T117R | T114R | Yes | No | Mutant | Mutant (Pang et al., 1997) |
| | R217C | R214C | No | ? | Δ Efficiency | — |
| | I219V | V216I | No | No | Polymorphism | — |
| | R265C | R265C | ? | ? | Δ Efficiency | — |
| | R265H | R265H | Yes | Yes | Δ Efficiency | — |
| | V326A | I326A | Yes | No | Δ Efficiency | — |
| | — | I326V | ? | ? | Polymorphism | — |
| | Q542L | Q552L | Yes | No | Mutant | — |
| | R659P | R672P | Yes | No | Mutant | — |
| | A681T | A694T | Yes | Yes | Polymorphism | — |
| MSH2 | G322D | G317D | Yes/No | ? | Δ Efficiency | Δ Efficiency (Drotschmann et al., 1999a) |
| | P622L | P640L | Yes | Yes | Mutant | Mutant (Drotschmann et al., 1999a) |
| | H639Y | H658Y | No | No | Polymorphism | — |
| ^{a}Classification: Mutant, indicates the gene conferred a mutation frequency that was not significantly different from the chromosomal null mutant lacking a complementing plasmid; Polymorphism, indicates the gene conferred a mutant frequency that was not different from the the null mutant strain that was complemented with the wild-type yeast gene; Δ Efficiency, indicates the gene confered a mutant frequency that is intermediate between the null mutant and the | | | | | | |
| # null mutant complemented with the wild-type yeast gene. | | | | | | |

? Indicates no information available.

TABLE 3

| | Mismatch | | MMR | |
|---|---|---|---|---|
| Reporter gene | repaired | Selection | defect | Reference |
| (GT)₁₆-URA3 | (GT)ₙ | ura⁻, FOA^{R} | 150 | Example 4 |
| | Insert/De- | | | |
| | lete loop | | | |
| hom3-10 | −1 frame- | THR⁺ | 500-750 | (Marischky et al., |
| | shift in | | | 1996) |
| | (T)₆ | | | |
| lys2::InsE-A₁₄ | −1 frame- | LYS⁺ | 10,000 | (Tran et al., 1997) |
| | shift in | | | |
| | (A)₁₄ | | | |
| lys2-ΔBgl | −1 frame- | LYS⁺ | 200-250 | (Greene & Jinks- |
| | shift near | | | Robertson, 1997) |
| | 683 | | | |
| lys2-ΔA746 | +1 frame- | LYS⁺ | 225-500 | (Harfe & Jinks- |
| | shift near | | | Roberstson, 1999) |
| | 746 | | | |
| his7-2 | +1, −2 | HIS⁺ | 85 | (Shcherbakova & |
| | frameshift | | | Kunkel, 1999) |
| | in (A)₇ | | | |
| CAN1 | wide | Canavanine^{R} | 25 | (Marischky et al., |
| | variety | | | 1996) |

TABLE 4
Additional human MLH1 and MSH2 variants examined and oligonucleotides used for making site-directed
mutations

| Variation | | Equivalent | | | | Restriction |
|---|---|---|---|---|---|---|
| in human | | substitution | Oligonucleotide sequence^{a} and SEQ ID# | | | site |
| MMR gene | | in yeast | [(S), sense and (A), anti-sense strand] | | | alteration^{b} |
| MLHI | I25F | I22F | 5′-GCTGCAGGTGAGATCTTTATATCCCCCGTAAATG-3′ | (S) | (SEQ ID 136) | +BgIlI |
| | | | 5′-CATTTACGGGGGATATAAAGATCTCACCTGCAGC-′3 | (A) | (SEQ ID 137) | |
| | I25T | I22T | 5′-GCTGCAGGTGAGATCACGATATCCCCCGTAAATG-3′ | (S) | (SEQ ID 138) | −EcoRV |
| | | | 5′-CATTTACGGGGGATATCGTGATCTCACCTGCAGC-′3 | (A) | (SEQ ID 139) | |
| | P28L | P25L | 5′-GGTGAGATCATAATATCACTAGTAAATGCTCTC-3′ | (S) | (SEQ ID 140) | +SpeI |
| | | | 5′-GAGAGCATTTACTAGTGATATTATGATCTCACC-′3 | (A) | (SEQ ID 141) | |
| | N64S | N61S | 5′-CAAATAACAGATAGTGGATCTGGAATTAATAAAG-3′ | (S) | (SEQ ID 142) | None^{c} |
| | | | 5′-CTTTATTAATTCCAGATCCACTATCTGTTATTTG-′3 | (A) | (SEQ ID 143) | |
| | I82I | T79I | 5′-GTGAGCGATTCACGATATCCAAATTACAAAAATTCG-3′ | (S) | (SEQ ID 144) | +EcoRV |
| | | | 5′-CGAATTTTTGTAATTTGGATATCGTGAATCGCTCAC-′3 | (A) | (SEQ ID 145) | |
| | K84E | K81E | 5′-GAGCGATTCACGACTTCCGAGTTACAAAAATTCGAAG-3′ | (S) | (SEQ ID 146) | −AatII |
| | | | 5′-CTTCGAATTTTTGTAACTCGGAAGTCGTGAATCGCTC-′3 | (A) | (SEQ ID 147) | |
| | A111V | A108V | 5′-GTATCTCACATGTGGTACGCGTCACAGTAACGAC-3′ | (S) | (SEQ ID 148) | +MluI |
| | | | 5′-GTCGTTACTGTGACGCGTACCACATGTGAGATAC-′3 | (A) | (SEQ ID 149) | |
| | V219L | V216L | 5′-GATAGGATTCGAACTCTGTTCAATAAATCTGTG-3′ | (S) | (SEQ ID 150) | +BstBI |
| | | | 5′-CACAGATTTATTGAACAGAGTTCGAATCCTATC-′3 | (A) | (SEQ ID 151) | |
| | 1262-del | 1262-del | 5′-CCATTTCATTAATTTTTTTCAATAATAGGCTAGTGAC-3′ | (S) | (SEQ ID 152) | −SpeI |
| | | | 5′-GTCACTAGCCTATTATTGAAAAAAATTAATGAAATGG-′3 | (A) | (SEQ ID 153) | |
| | L653R | L666R | 5′-CCATCTCTGGTCAAGCGGCCATTTTTTATATATCGCC-3′ | (S) | (SEQ ID 154) | +EaeI |
| | | | 5′-GGCGATATATAAAAAATGGCCGCTTGACCAGAGATGG-′3 | (A) | (SEQ ID 155) | |
| | P654L | P667L | 5′-CTCTGGTCAAGCTTCTATTTTTTATATATCGCCTG-3′ | (S) | (SEQ ID 156) | +HindIII |
| | | | 5′-CAGGCGATATATAAAAAATAGAAGCTTGACCAGAG-′3 | (A) | (SEQ ID 157) | |
| | R659L | R672L | 5′-CCATTTTTTATATATCTCCTAGGTAAAGAAGTTG-3′ | (S) | (SEQ ID 158) | +AvrII |
| | | | 5′-CAACTTCTTTACCTAGGAGATATATAAAAAATGG-′3 | (A) | (SEQ ID 159) | |
| | E663D | E676D | 5′-CCTGGGTAAAGACGTCGATTGGGAGGATGAAC-3′ | (S) | (SEQ ID 160) | +AatII |
| | | | 5′-GTTCATCCTCCCAATCGACGTCTTTACCCAGG-′3 | (A) | (SEQ ID 161) | |
| | H718Y | H733Y | 5′-TATCCTCATTACTAGAATACGTCCTCTTCCCTTG-3′ | (S) | (SEQ ID 162) | −XmnI |
| | | | 5′-AAGGGAAGAGGACGTATTCTAGTAATGAGGATA-′3 | (A) | (SEQ ID 163) | |
| | L729V | L744V | 5′-GTATCAAACGAAGGTTCGTGGCCCCTAGACAC-3′ | (S) | (SEQ ID 164) | None^{c} |
| | | | 5′-GTGTCTAGGGGCCACGAACCTTCGTTTGATAC-′3 | (A) | (SEQ ID 165) | |
| | K751R | K764R | 5′-CCACCTTCCGGATCTATACAGAGTTTTTGAGAG-3′ | (S) | (SEQ ID 166) | −BgIII |
| | | | 5′-CTCTCAAAAACTCTGTATAGATCCGGAAGGTGG-′3 | (A) | (SEQ ID 167) | |
| | R755W | R768W | 5′-CAAAGTTTTTGAGTGGTGTAAACT-3′ | (S) | (SEQ ID 168) | None^{c} |
| | | | 5′-AGTTTACACCACTCAAAAACTTTG-′3 | (A) | (SEQ ID 169) | |
| MSH2 | R524P | R542P^{d} | 5′-ATGGTTGGTGCATGCCGTTGACACGTAATGAC-3′ | (S) | (SEQ ID 170) | +SphI |
| | | | 5′-GTCATTACGTGTCAACGGCATGCACCAACCAT-′3 | (A) | (SEQ ID 171) | |

^{a}Sense and antisense oligonucleotides were employed for site-directed mutations in yeast MMR genes as described in Example 1.
^{b}Restriction site alterations are silent, except for the indicated amino acid substitutions. +, restriction site additon; −, restriction site loss.
^{c}Alteration screened by DNA sequencing.
^{d}Alteration made by overlap extension PCR as described in Example 17.

TABLE 5
MLH1 missense changes that impair function from prospective mutational analysis

| | | | Corresponding | |
|---|---|---|---|---|
| Amino acid | | | human alteration | |
| position | Variant Isolated^{a} | | (if yeast portion | |
| in hMLH 1 | and SEQ ID # | | of hybrid) | Association with cancer |
| 11 | yL8H | (SEQ ID 193) | L11H | |
| 19 | yI16F | (SEQ ID 194) | I19F | |
| 33 | yK30N | (SEQ ID 195) | K33N | |
| 38 | yN35D | (SEQ ID 196) | N38D | |
| 38 | yN35S | (SEQ ID 197) | N385 | |
| 40 | yI37T | (SEQ ID 198) | L40T | |
| 41 | hD41G | (SEQ ID 199) | — | |
| 44 | yA41V | (SEQ ID 200) | S44V | Alteration at same aa as in HNPCC patient |
| 45 | yT42I | (SEQ ID 201) | T451 | |
| 49 | hV49A | (SEQ ID 202) | — | |
| 52 | hK52I | (SEQ ID 203) | — | |
| 52 | yK49E | (SEQ ID 204) | K52E | |
| 53 | hE53V | (SEQ ID 205) | — | |
| 54 | hG54R | (SEQ ID 206) | — | |
| 55 | yG52R | (SEQ ID 207) | G55R | |
| 55 | hG55D | (SEQ ID 208) | — | |
| 57 | hK57E | (SEQ ID 209) | — | |
| 60 | hQ60P | (SEQ ID 210) | — | |
| 61 | yI58K | (SEQ ID 211) | I61K | |
| 65 | yG62R | (SEQ ID 212) | G65R | |
| 67 | hG67V | (SEQ ID 213) | — | Alteration at same aa as in HNPCC patient |
| 67 | hG67E | (SEQ ID 214) | — | Identical to alteration in HNPCC patient |
| 72 | hD72V | (SEQ ID 215) | — | |
| 73 | hL73Q | (SEQ ID 216) | — | |
| 77 | hC77R | (SEQ ID 217) | — | Identical to alteration in HNPCC patient |
| 77 | hC77G | (SEQ ID 218) | — | Alteration at same aa as in HNPCC patient |
| 78 | hE78V | (SEQ ID 219) | — | |
| 80 | hF80L | (SEQ ID 220) | — | |
| 81 | hT81M | (SEQ ID 221) | — | |
| 84 | hK84R | (SEQ ID 222) | — | Alteration at same aa as in HNPCC patient |
| 84 | hK84E | (SEQ ID 223) | — | Identical to alteration in HNPCC patient |
| 85 | hL85S | (SEQ ID 224) | — | |
| 103 | hA103P | (SEQ ID 225) | — | |
| 116 | hT116S | (SEQ ID 226) | — | |
| 118 | hK118I | (SEQ ID 227) | — | |
| 133 | hG133E | (SEQ ID 228) | — | |
| 139 | yP136H | (SEQ ID 229) | P139H | |
| 143 | yA140V | (SEQ ID 230) | A143V | |
| 147 | yG144S | (SEQ ID 231) | G147S | |

^{a}Amino acids are abbreviated by single letter code. Numbering corresponds to the position in the native y, yeast or h, human MLH1 protein.

TABLE 6
Functional consequence of missense alterations at
hMLH1 codon 44 (S44)

| Consequence for | | |
|---|---|---|
| function in MMR | Amino acid alteration | |
| No loss of function | S44S | (SEQ ID NO: 179) |
| (Polymorphism) | S44A | (SEQ ID NO: 180) |
| Loss of function | S44R | (SEQ ID NO: 181) |
| (Mutation) | S44C | (SEQ ID NO: 182) |
| | S44Q | (SEQ ID NO: 183) |
| | S44H | (SEQ ID NO: 184) |
| | S44I | (SEQ ID NO: 185) |
| | S44L | (SEQ ID NO: 186) |
| | S44F | (SEQ ID NO: 187) |
| | S44P | (SEQ ID NO: 188) |
| | S44T | (SEQ ID NO: 189) |
| | S44W | (SEQ ID NO: 190) |
| | S44Y | (SEQ ID NO: 191) |
| | S44V | (SEQ ID NO: 192) |

Saturation mutagenesis at hMLH1 codon 44 was described in Example 19.
Codon changes corresponding to hMLH1 S44M, S44N, S44K, S44D, S44E, and S44G are tested in vivo according to Examples 1 and 5.

Abbreviations:

CRC: colorectal cancer

HNPCC: hereditary nonpolyposis colorectal cancer

MMR: DNA mismatch repair

PCR: polymerase chain reaction

NY: a codon at position N in a gene (N denoting the number of the codon, where the ATG translation initiation codon is assigned number 1) which encodes the amino acid X (encoding one of the twenty amino acids, the symbols for which are listed below).

XNY: a codon at position N in a gene (N denoting the number of the codon, where the ATG translation initiation codon is assigned number 1) in which the codon for amino acid X (encoding one of the twenty amino acids, the symbols for which is below) has been changed to codon Y (again represented by one of the twenty symbols below).

A: the amino acid alanine

C: the amino acid cysteine

D: the amino acid aspartic acid

E: the amino acid glutamic acid

F: the amino acid phenylalanine

G: the amino acid glycine

H: the amino acid histidine

I: the amino acid isoleucine

K: the amino acid lysine

L: the amino acid leucine

M: the amino acid methionine

N: the amino acid asparagine

P: the amino acid proline

Q: the amino acid glutamine

R: the amino acid arginine

S: the amino acid serine

T: the amino acid threonine

V: the amino acid valine

W: the amino acid tryptophan

Y: the amino acid tyrosine

### REFERENCES

Altschul, S. F., Madden, T. L., Schaffer, A. A., Zhang, J., Zhang, Z., Miller, W. & Lipman, D. J. (1997). Gapped BLAST and PSI-BLAST: a new generation of protein database search programs. Nucleic Acids Res. 25, 3389-3402.

Ban, C. & Yang, W. (1998). Crystal Structure and ATPase Activity of MutL: Implications for DNA Repair and Mutagenesis. Cell 95, 541-552.

Bitter, G. A. (1998). Function of hybrid human-yeast cyclin dependent kinases in Saccharomyces cerevisiae. Mol. and Gen. Genet. 260, 120-130.

Boeke, J. D., Lacroute, F. & Fink, G. (1984). A positive selection for mutants lacking orotidine-5′-phosphate decarboxylase activity in yeast: 5-fluoro-orotic acid resistance. Mol. Gen. Genet. 197, 345-346.

Borreson, A. L., Lothe, R. A., Meling, G. I., Lystad, S., Morrison, P., Lipford, J., Kane, M. F., Rognum, T. O. & Kolodner, R. D. (1995). Somatic mutations in the hMSH2 gene in microsatellite unstable colorectal carcinomas. Hum. Mol. Genet. 4(11), 2065-2072.

Bronner, C. E., Baker, S. M., Morrison, P. T., Warren, G., Smith, L. G., Lescoe, M. K., Kane, M., Earabino, C., Lipford, J., Lindblom, A., Tannergard, P., Bollag, R. J., Godwin, A. R., Ward, D. C., Nordenskjold, M., Fishel, R., Kolodner, R. & Liskay, R. M. (1994). Mutation in the DNA mismatch repair gene homologue hMLH1 is associated with hereditary non-polyposis colon cancer. Nature 368, 258-261.

Buerstedde, J.-M., Alday, P., Torhorst, J., Weber, W., Muller, H. & Scott, R. (1995). Detection of new mutations in six out of ten Swiss HNPCC families by genomic sequencing of the hMSH2 and hMLH1 genes. J. Med. Genet. 32, 909-912.

Chen, C. & Kolodner, R. D. (1999). Gross chromosomal rearrangements in Saccharomyces cerevisiae replication and recombination defective mutants. Nature Genetics 23(1), 81-85.

Clark, A. B., Cook, M. E., Tran, H. T., Gordenin, D. A., Resnick, M. & Kunkel, T. A. (1999). Functional analysis of human MutSα and MutSβ complexes in yeast. Nucleic Acids Res. 27(3), 736-742.

Cunningham, J. M., Kim, C.-Y., Christensen, E. R., Tester, D. J., Parc, Y., Burgart, L. J., Hailing, K. C., McDonnell, S. K., Schaid, D. J., Vockley, C. W., Kubly, V., Nelson, H., Michels, V. V. & Thibodeau, S. N. (2001). The frequency of hereditary defective mismatch repair in a prospective series of unselected colorectal carcinomas. Am. J. Hum. Genet. 69, 780-790.

Das Gupta, R. & Kolodner, R. D. (2000). Novel dominant mutations in Saccharomyces cerevisiae MSH6. Nature Genetics 24, 53-56.

Drotschmann, K., Clark, A. B. & Kunkel, T. A. (1999a). Mutator phenotypes of common polymorphisms and missense mutations in MSH2. Current Biology 9, 907-910.

Drotschmann, K., Clark, A. B., Tran, H. T., Resnick, M. A., Gordenin, D. A. & Kunkel, T. A. (1999b). Mutator phenotypes of yeast strains heterozygous for mutations in the MSH2 gene. Proc. Natl. Acad. Sci. USA 96(March), 2970-2975.

Fishel, R., Lescoe, M. K., Rao, M. R. S., Copeland, N. G., Jenkins, N. A., Garber, J., Kane, M. & Kolodner, R. (1993). The Human Mutator Gene Homolog MSH2 and Its Association with Hereditary Nonpolyposis Colon Cancer. Cell 75, 1027-1038.

Fishel, R. & Wilson, T. (1997). MutS homologs in mammalian cells. Curr. Opin. Genet. Dev. 7, 105-113.

Froggatt, N. J., Brasset, C., Koch, D. J., Evans, D. G., Hodgson, S. V., Popnder, B. A. & Maher, E. R. (1996). Mutation Screening of MSH2 and MLH1 mRNA in hereditary non-polyposis colon cancer syndrome. J. Med. Genet. 33(9), 726-730.

Gordenin, D. A., Proscyavivhus, Y. Y., Malkova, A. L., Trofimova, M. V. & Peterzen, A. (1991). Yeast mutants with increased bacterial transposon Tn5 excision. Yeast 7, 37-50.

Greene, C. N. & Jinks-Robertson, S. (1997). Frameshift intermediates in homopolymer runs are removed efficiently by yeast mismatch repair proteins. Mol. Cell. Biol. 17, 284-2850.

Hackman, P., Tannergard, P., Osei-Mensa, S., Chen, J., Kane, M. F., Kolodner, R., Lambert, B., Hellgren, D. & Lindblom, A. (1997). A human compound heterozygote for two MLH1 missense mutations. Nature Genetics 17, 135-136.

Han, H.-J., Yuan, Y., Ku, J.-L., Oh, Y. J., Won, Y. J., Kang, K. J., Kim, K. Y., Kim, S., Kim, C. Y., Kim, J.-P., Oh, N.-G., Lee, K. H., Choe, K. J. N., Y. & Park, J. G. (1996). Germline Mutations of hMLH1 and hMSH2 Genes in Korean Hereditary Nonpolyposis Colorectal Cancer. J. Natl. Cancer Inst. 88(18), 14317-1319.

Hangaishi, A., Ogawa, S., Mitani, K., Hosoya, N., Chiba, S., Yazaki, Y. & Hirai, H. (1997). Mutations and loss of expression of a mismatch repair gene, hMLH1, in leukemia and lymphoma cell lines. Blood 89(5), 1740-1747.

Harfe, B. D. & Jinks-Roberstson, S. (1999). Removal of Frqameshift Intermediates by Mismatch Repair Proteins in Saccharomyces cerevisiae. Mol. Cell. Biol. 19(7), 4766-4773.

Herman, J. G., Umar, A., Polyak, K., Graff, J. R., Ahuja, N., Issa, J. P., Markowitz, S., Willson, J. K. V., Hamilton, S. R., Kinzler, K. W., Kane, M. F., Kolodner, R. D., Vogelstein, B., Kunkel, T. & Baylin, S. B. (1998). Incidence and functional consequences of hMLH1 promoter hypermethylation in colorectal carcinoma. Proc. Natl. Acad. Sci. USA 95, 6870-6875.

Hoffman, C. S. & Winston, F. (1987). A ten-minute DNA preparation from yeast efficiently releases autonomous plasmids for transformation of Escherichia coli. Gene 57, 267-272.

Hutter, P., Couturier, A., Scott, R. J., Alday, P., Delozier-Blanchet, C., Cachat, F., Antonarakis, S. E., Joris, F., Gaudin, M., D'Amato, L. & Buerstedde, J.-M. (1996). Complex genetic predisposition to cancer in an extended HNPCC family with an ancestral hMLH1 mutation. J. Med. Genet. 33, 636-640.

Ishioka, C., Frebourg, T., Yan, Y. X., Vidal, M., Friend, S. H., Schmidt, S. & Iggo, R. (1993). Screening patients for heterozygous p53 mutations using a functional assay in yeast. Nat. Genet. 5(2), 124-129.

Ito, H., Fukuda, Y., Murata, K. & Kimura, A. (1983). Transformation of intact yeast cells treated with alkali cations. Journal of Bacteriology 153, 163-168.

Jakubowska, A., Gorski, B., Kurzaski, G., Debniak, T., Hadaczek, P., Cybulski, C., Kladny, J., Oszurek, O., Scott, R. J. & Lubinski, J. (2001). Optimization of experimental conditions for RNA-based sequencing of MLH1 and MSH2 genes. Human Mutation 17, 52-60.

Jiricny, J. & Nystrom-Lahti, M. (2000). Mismatch Repair Defects in Cancer. Curr. Opin. Genet. & Dev. 10, 157-161.

Kinzler, K. W. & Vogelstein, B. (1996). Lessons from Hereditary Colorectal Cancer. Cell 87(2), 159-170.

Kolodner, R. D. (2000). Guarding Against Mutations. Nature 407, 687-689.

Kolodner, R. D. & Marisischky, G. T. (1999). Eukaryotic DNA mismatch Repair. Current Opinion in Genetics and Development 9, 89-96.

Lamers, M. H., Perrakis, A., Enzlin, J. H., Winterwerp, H. H. K., de Wind, N. & Sixma, T. K. (2000). The Crystal Structure of DNA Mismatch Repair Protein MutS binding to a G:T Mismatch. Nature 407, 711-717.

Leach, F. S., Nicolaides, N. C., Papadopoulos, N., Liu, B., Jen, J., Parsons, R., Peltomaki, P., Sistonen, P., Aaltonen, L. A., Nystrom-Lahti, M., Guan, X. Y., Zhang, J., Meltzer, P. S., Yu, J.-W., Kao, F.-T., Chen, D. J., Cerosaletti, K. M., Fournier, R. E. K., Todd, S., Lewis, T., Leach, R. J., Naylor, S. L., Weissenbach, J., Mecklin, J.-P., Jarvinen, H., Petersen, G. M., Hamilton, S. R., Green, J., Jass, J., Watson, P., Lynch, H. T., Trent, J. M., Chapell, A. d. l., Kinzler, K. W. & Vogelstein, B. (1993). Mutations of a mutS Homolog in Hereditary Nonpolyposis Colorectal Cancer. Cell 75, 1215-1225.

Liu, B., Nicolaides, N. C., Markowitz, S., Parsons, R. E., Papadopoulous, N., Nicolaides, N. C., Lynch, H. T., Watson, P., Jass, J. R., Dunlop, M., Wyllie, A., Peltomaki, P., Chapelle, A. d. l., Hamilton, S. R., Vogelstein, B. & Kinzler, K. W. (1996). Analysis of mismatch repair genes in hereditary non-polyposis colorectal cancer patients. Nature Medicine 2, 169-174.

Liu, B., Nicolaides, N. C., Markowitz, S., Willson, J. K. V., Parsons, R. E., Jen, J., Papadopoulos, N., Peltomaki, P., de la Chapelle, A., Hamilton, S. R., Kinzler, K. W. & Vogelstein, B. (1995). Mismatch repair gene defects in sporadic colorectal cancers with microsatellite instability. Nature Genetics 9, 48-55.

Liu, H.-H., Cartegni, L., Zhang, M. Q. & Krainer, A. R. (2001). A mechanism for exon skipping caused by nonsense or missense mutations in BRCA1 and other genes. Nature Genetics 27(1), 55-58.

Lowsky, R., DeCoteau, J. F., Reitmair, A. H., Ichinohasama, R., Dong, W. F., Wu, Y., Mak, T. W., Kadin, M. E. & Minden, M. D. (1997). Defects in the mismatch repair gene MSH2 are implicated in the development of murine and human lymphoblastic lymphomas and are associated with the aberrant expression of rhombotin-2 (Lmo-2) and Tal-1 (SCL). Blood 89(7), 2276-2282.

Maliaka, Y. K., Chudina, A. P., Belev, N. F., Alday, P., Bochkov, N. P. & Buerstedde, J.-M. (1996). CpG dinucleotides in the hMSH2 and hMLH1 genes are hotspots for HNPCC mutations. Hum. Genet. 97, 251-255.

Maniatis, T., Fritsch, E. F. & Sambrook, J. (1989). Molecular Cloning. A Laboratory Manual, 2nd edition, Cold Spring Harbor Laboratory Press.

Marischky, G. T., Filosi, N., Kane, M. F. & Kolodner, R. (1996). Redundancy of Saccharomyces cerevisiae MSH3 and MSH6 in MSH2-dependent mismatch repair. Genes Dev. 10, 407-420.

Miyaki, M., Konishi, M., Muraoka, M., Kikuchi-Yanoshita, R., Tanaka, K., Iwama, T., Mori, T., Koike, M., Ushio, K., Chiba, M., Nomizu, S. & Utsunomiya, J. (1995). Germ line mutations of hMSH2 and hMLH1 genes in Japanese families with hereditary nonpolyposis colorectal cancer (HNPCC): usefulness of DNA analysis for screening and diagnosis of HNPCC patients. J. Mol. Med. 73, 515-520.

Moslein, G., Tester, D. T., Lindor, N. M., Honchel, R., Cunningham, J. M., French, A. J., Halling, K. C., Schwab, M., Goretski, P. & Thibodeau, S. N. (1996). Microsatellite instability and mutation analysis of hMSH2 and hMLH1 in patients with sporadic, familial and hereditary colorectal cancer. Hum. Mol. Genet. 9, 1245-1252.

Mumberg, D., Muller, R. & Funk, M. (1994). Regulatable promoters of Saccharomyces cerevisiae: comparison of transcriptional activity and their use for heterologous expression. Mol. Gen. Genet. 22(25), 5767-5768.

Nicolaides, N. C., Littman, S. J., Modrich, P., Kinzler, K. W. & Vogelstein, B. (1998). A Naturally Ocurring hPMS2 Mutatin Can Confer a Dominant Negative Mutator Phenotype. Mol. Cell. Biol. 18(3), 1635-1641.

Nicolaides, N. C., Papadopoulos, N., Liu, B., Wei, Y.-f., Carter, K. C., Ruben, S. M., Rosen, C. A., Hazeltine, W. A., Fleischman, R. D., Fraser, C. M., Adams, M. D., Venter, J. C., Dunlop, M. G., Hamilton, S. R., Petersen, G. M., Chapelle, A. d. l., Vogelstein, B. & Kinzler, K. W. (1994). Mutations of two PMS homologues in hereditary nonpolyposis colon cancer. Nature 371, 75-80.

Nystrom-Lahti, M., Wu, Y., Moisio, A.-L., Hofstra, R. M. W., Osinga, J., Mecklin, J.-P., Jarvinen, H. J., Leisti, J., Buys, C., de la Chapelle, A. & Peltomaki, P. (1996). DNA mismatch repair gene mutations in 55 kindreds with verified or putative hereditary non-polyposis colorectal cancer. Hum. Mol. Genet. 5, 763-769.

Obmolova, G., Ban, C., Hsieh, P. & Yang, W. (2000). Crystal Structure of Mismatch Repair Protein MutS and its Complex with a Substrate DNA. Nature 407, 703-710.

Pang, Q., Prolla, T. A. & Liskay, R. M. (1997). Functional Domains of the Saccharomyces cerevisiae Mlh1p and Pms1p DNA Mismatch Repair Proteins and Their Relevance to Human Hereditary Nonpolyposis Colorectal Cancer—Associated Mutations. Mol. Cell. Biol. 17(8), 4465-4473.

Papadopoulos, N. & Lindblom, A. (1997). Molecular Basis of HNPCC: Mutations of MMR Genes. Human Mutation 10, 89-99.

Papadopoulos, N., Nicoolaides, N. C., Wei, Y.-F., Ruben, S. M., Carter, K. C., Rosen, C. A., Hazeltine, W. A., Fleischman, R. D., Fraser, C. M., Adams, M. D., Venter, J. C., Hamilton, S. R., Petersen, G. M., Watson, P., Lynch, H. T., Peltomaki, P., Mecklin, J.-P., Chapelle, A. d. l., Kinzler, K. W. & Vogelstein, B. (1994). Mutation of a mutL Homolog in Hereditary Colon Cancer. Science 263, 1625-1629.

Peltomaki, P. & Chapelle, A. d. l. (1997). Mutations predisposing to hereditary nonpolyposis colorectal cancer. Adv. Cancer Res. 71, 93-119.

Peltomaki, P., Vasen, H. & HNPCC, T. I. C. G. o. (1997). Mutations Predisposing to Hereditary Nonpolyposis Colorectal Cancer: Database and Results of a Collaborative Study. Gastroenterology 113, 1146-1158.

Polaczek, P., Putzke, A. P., Leong, K. & Bitter, G. A. (1998). Functional genetic tests of DNA mismatch repair protein activity in Saccharomyces cerevisiae. Gene 213, 159-167.

Scharer, E. & Iggo, R. (1992). Mammalian p53 can function as a transcription factor in yeast. Nucleic Acids Res. 20, 1539-1545.

Semba, S., Yokozaki, H., Yamamoto, S., Yasui, W. & Tahara, E. (1996). Microsatellite instability in in precancerous lesions and adenocarcinomas of the stomach. Cancer 77, 1620-1627.

Shcherbakova, P. V., Hall, M. C., Lewis, M. S., Bennett, S. E., Martin, K. J., Bushel, P. R., Afshari, C. A. & Kunkel, T. A. (2001). Inactivation of DNA Mismatch Repair by Increased Expression of Yeast MLH1. Mol Cell. Biol. 21(3), 940-951.

Shcherbakova, P. V. & Kunkel, T. (1999). Mutator Phenotypes Conferred by MLH1 Overexpression and by Heterozygosity for mlh1 Mutations. Mol. Cell. Biol. 19(4), 3177-3183.

Shimodaira, H., Filosi, N., Shibata, H., Suzuki, T., Radice, P., Kanamaru, R., Friend, S. H., Kolodner, R. D. & Ishioka, C. (1998). Functional analysis of human MLH1 mutations in Saccharomyces cerevisiae. Nature Genetics 19, 384-389.

Sikorski, R. S. & Hieter, P. (1989). A system of shuttle vectors and yeast host strains designed for efficient manipulation of DNA in Saccharomyces cerevisiae. Genetics 122(1), 19-27.

Strand, M., Prolla, T. A., Liskay, R. M. & Petes, T. D. (1993). Destabilization of tracts of simple repetitive DNA in yeast by mutations affecting DNA mismatch repair. Nature 365, 274-276.

Studamire, B., Price, G., Sugawara, N., Haber, J. & Alani, E. (1999). Separation-of-Function Mutations in Saccharomyces cerevisiae MSH2 That Confer Mismatch Repair Defects but Do Not Affect Nonhomologous-Tail Removal during Recombination. Mol. Cell. Biol. 19(11), 7558-7567.

Syngal, S., Fox, E. A., Li, C., Dovidio, M., Eng, C., Kolodner, R. D. & Garber, J. E. (1999). Interpretation of genetic test results for hereditary nonpolyposis colorectal cancer: implications for clinical predisposition testing. JAMA 282(3), 247-253.

Tannergard, P., Lipford, J. R., Koldner, R., Frodin, J. E., Nordenskjold, M. & A. Lindblom. (1995). Missense mutations in the MLH1 gene in Swedish HNPCC families. Cancer Research 55, 6092-6096.

Terdiman, J. P., Gum Jr., J. R., Conrad, P. G., Miller, G. A., Weinberg, V., Crawley, S. C., Levin, T. R., Reeves, C., Schmitt, A., Hepburn, M., Sleisenger, M. H. & Kim, Y. S. (2001). Efficient detection of hereditary nonpolyposis colorectal cancer gene carriers by screening for tumor microsatellite instability before germline testing. Gastroenterology 120, 21-30.

Tomlinson, I. P. M., Beck, N. E., Homfray, T., Harocopos, C. J. & Bodmer, W. F. (1997). Germline HNPCC gene variants have little influence on the risk for sporadic colorectal cancer. J. Med. Genet. 34, 39-42.

Tran, H. T., Keen, J. D., Kricker, M., Resnick, M. A. & Gordenein, D. A. (1997). Hypermutability of Homonucleotide Runs in Mismatch Repair and DNA Polymerase Proofreading Yeast Mutants. Mol. Cell. Biol. 17(5), 2859-2865.

Vasen, H. F., Mecklin, J. P., Khan, P. M. & Lynch, H. T. (1991). The International Collaborative Group on Hereditary Non-Polypolposis Colorectal Cancer (ICG-HNPCC). Dis. Colon Rectum 34(5), 424-425.

All publications and patent applications mentioned in this specification are herein incorporated by reference to the same extent as if each individual publication or patent application was specifically and individually indicated to be incorporated by reference.

The invention now being fully described, it will be apparent to one of ordinary skill in the art that many changes and modifications can be made thereto without departing from the spirit or scope of the invention as described herein.

1. A method for distinguishing efficiency polymorphisms from inactivating mutations and silent polymorphisms in a human gene encoding a protein involved in DNA mismatch repair, comprising:
expressing a test genetic sequence in a null yeast host in which the native yeast DNA mismatch repair gene which is the orthologue of said human gene has been deleted or modified to inactivate its function in DNA mismatch repair, and
comparing the mutation rate of a reporter gene in the null yeast host of the previous step with the mutation rate of the same reporter gene in (1) the null yeast host which has not been transformed to express said test genetic sequence and (2) the null yeast host which has been transformed to express the yeast orthologue of said human gene, or a hybrid human-yeast orthologue of said human gene, in order to determine if the test genetic sequence is an efficiency polymorphism.

2. The method of claim 1, wherein the test genetic sequence is a yeast orthologue variant of the human gene sequence or a human-yeast hybrid sequence of said variant.

3. The method of claim 1, wherein the human gene involved in DNA mismatch repair is selected from the group consisting of the hMSH2 (SEQ ID NO: 3), hMSH3 (SEQ ID NO: 4), hMSH4 (SEQ ID NO: 5), hMSH6 (SEQ ID NO: 6), hMLH1 (SEQ ID NO: 7), hMLH3 (SEQ ID NO: 8), hPMS1 (SEQ ID NO: 9) and hPMS2 (SEQ ID NO: 10) genes.

4. The method of claim 3, wherein said human gene involved in DNA mismatch repair is selected from the group consisting of the hMLH1 (SEQ ID NO: 7) and hMSH2 (SEQ ID NO: 3) genes.

5. The method of claim 4, which is used to distinguish among the following variants in the hMLH1 gene: S44A (SEQ ID NO: 232), S44F (SEQ ID NO: 126), S44R (SEQ ID NO: 233), S44C (SEQ ID NO: 234), S44Q (SEQ ID NO: S35), S44H (SEQ ID NO: 236), S441 (SEQ ID NO: 237), S44L (SEQ ID NO: 238), S44P (SEQ ID NO: 239), S44T (SEQ ID NO: 240), S44W (SEQ ID NO: 241), S44Y (SEQ ID NO: 242), S44V (SEQ ID NO: 243), G67R (SEQ ID NO: 244), I68N (SEQ ID NO: 245), E102K (SEQ ID NO: 246), I107R (SEQ ID NO: 247), T117R (SEQ ID NO: 248), Q542L (SEQ ID NO: 249), R659P (SEQ ID NO: 250), R217C (SEQ ID NO: 251), R265C (SEQ ID NO: 252), R265H (SEQ ID NO: 253), V3261 (SEQ ID NO: 254), V326A (SEQ ID NO: 127), Q62K (SEQ ID NO: 255), R69K (SEQ ID NO: 256), I219V (SEQ ID NO: 257), A681T (SEQ ID NO: 258), I25F (SEQ ID NO: 259), I25T (SEQ ID NO: 260), P28L (SEQ ID NO: 261), N64S (SEQ ID NO: 262), A111V (SEQ ID NO: 263), 1262-del (SEQ ID NO: 264), L653R (SEQ ID NO: 265), P654L (SEQ ID NO: 266), R659L (SEQ ID NO: 267), T821 (SEQ ID NO: 268), K84E (SEQ ID NO: 269), R755W (SEQ ID NO: 270), S93G (SEQ ID NO: 271), I219L (SEQ ID NO: 272), E663D (SEQ ID NO: 273), H718Y (SEQ ID NO: 274), L729V (SEQ ID NO: 275), K75 IR (SEQ ID NO: 276),

6. The method of claim 5, which is used to identify any of the following variants as an efficiency polymorphism: R217C (SEQ ID NO: 251), R265C (SEQ ID NO: 252), R265H (SEQ ID NO: 253), V326A (SEQ ID NO: 127), Q62K (SEQ ID NO: 255), R69K (SEQ ID NO: 256), I25F (SEQ ID NO: 259), I25T (SEQ ID NO: 260), P28L (SEQ ID NO: 261), N64S (SEQ ID NO: 262), A111V (SEQ ID NO: 263), 1262-del (SEQ ID NO: 264), L653R (SEQ ID NO: 265), P654L (SEQ ID NO: 266), R659L (SEQ ID NO: 267).

7. The method of claim 5, which is used to identify any of the following variants as an inactivating mutation: S44F (SEQ ID NO: 126), S44R (SEQ ID NO: 233), S44C (SEQ ID NO: 234), S44Q (SEQ ID NO: 235), S44H (SEQ ID NO: S36), S441 (SEQ ID NO: 237), S44L (SEQ ID NO: 238), S44P (SEQ ID NO: 239), S44T (SEQ ID NO: 240), S44W (SEQ ID NO: 241), S44Y (SEQ ID NO: 242), S44V (SEQ ID NO: 243), G67R (SEQ ID NO: 244), 168N (SEQ ID NO: 245), E102K (SEQ ID NO: 246), 1107R (SEQ ID NO: 247), T117R (SEQ ID NO: 248), Q542L (SEQ ID NO: 249), R659P (SEQ ID NO: 250) T821 (SEQ ID NO: 251), K84E (SEQ ID NO: 252) and R755W (SEQ ID NO: 270).

8. The method of claim 5, which is used to identify any of the following variants as a silent polymorphism: S44A (SEQ ID NO: 232), I219V (SEQ ID NO: 257), A681T (SEQ ID NO: 258), S93G (SEQ ID NO: 271), V3261 (SEQ ID NO: 254), I219L (SEQ ID NO: 272), E663D (SEQ ID NO: 273), H718Y (SEQ ID NO: 274), L729V (SEQ ID NO: 275) and K751R (SEQ ID NO: 276).

9. The method of claim 4, which is used to identify the following efficiency polymorphisms in the hMSH2 gene: G322D (SEQ ID NO: 66) and R524P (SEQ ID NO: 277).

10. A method for identifying defects in genes involved in DNA mismatch repair, comprising:
producing by random mutagenesis or in vitro mutagenic DNA synthesis a pool of
mutagenized DNA molecules corresponding to said gene, or a region of the coding sequence therof,
expressing said pool of DNA molecules in yeast host cells,
growing the yeast host cells, and
screening for yeast clones which exhibit a deficiency in DNA mismatch repair.

11. The method of claim 10, which is used to evaluate a DNA mismatch repair gene selected from the group consisting of the hMSH2 (SEQ ID NO: 3), hMSH3 (SEQ ID NO: 4), hMSH4 (SEQ ID NO: 5), hMSH6 (SEQ ID NO: 6), hMLH1 (SEQ ID NO: 7), hMLH3 (SEQ ID NO: 8), hPMS1 (SEQ ID NO: 9) and hPMS2 (SEQ ID NO: 10) genes.

12. The method of claim 11, wherein said human gene involved in DNA mismatch repair is selected from the group consisting of the hMLH1 (SEQ ID NO: 7) and hMSH2 (SEQ ID NO: 3) genes.

13. The method of claim 10 which is used to identify the following as inactivating mutations in hMLH1: L11H (SEQ ID NO: 278), I19F (SEQ ID NO: 279), K33N (SEQ ID NO: 280), N38D (SEQ ID NO: 281), N38S (SEQ ID NO: 282), L40T (SEQ ID NO: 283), D41G (SEQ ID NO: 284), S44V (SEQ ID NO: 243), T45I (SEQ ID NO: 285), V49A (SEQ ID NO: 286), K52I (SEQ ID NO: 287), K52E (SEQ ID NO: 288), E53V (SEQ ID NO: 289), G54R (SEQ ID NO: 290), G55R (SEQ ID NO: 291), G55D (SEQ ID NO: 292), K57E (SEQ ID NO: 293), Q60P (SEQ ID NO: 294), I61K (SEQ ID NO: 295), G65R (SEQ ID NO: 296), G67V (SEQ ID NO: 297), G67E (SEQ ID NO: 298), D72V (SEQ ID NO: 299), L73Q (SEQ ID NO: 300), C77R (SEQ ID NO: 301), C77G (SEQ ID NO: 302), E78V (SEQ ID NO: 303), F80L (SEQ ID NO: 304), T81M (SEQ ID NO: 305), K84R (SEQ ID NO: 306), K84E (SEQ ID NO: 269), L85S (SEQ ID NO: 307), A103P (SEQ ID NO: 308), T116S (SEQ ID NO: 309), K118I (SEQ ID NO: 10), G133E (SEQ ID NO: 311), P139H (SEQ ID NO: 312), A143V (SEQ ID NO: 313), G147S (SEQ ID NO: 314).

14. A method for determining whether a genetic sequence in an individual is associated with a defect in DNA mismatch repair, comprising comparing said genetic sequence with a genetic information database that has been compiled by use of the method of claims 1 or 10.

15. The method of claim 14, which is used to evaluate predisposition to the onset of cancer in a human.

16. A DNA molecule encoding a yeast protein involved in DNA mismatch repair in which a portion of the coding sequence has been replaced with the homologous coding sequence of the human orthologue to produce a hybrid human-yeast gene, such that the protein expression product of the hybrid gene retains function in DNA mismatch repair in vivo.

17. The method of claim 16, wherein said yeast DNA molecule encoding a yeast protein involved in DNA mismatch repair is selected from the group consisting of the MLH1 (SEQ ID NO: 1) and MSH2 (SEQ ID NO: 2) genes and said human orthologue is selected from the group consisting of the hMLH1 (SEQ ID NO: 7) and hMSH2 (SEQ ID NO: 3) genes.

18. An efficiency polymorphism in a variant of a DNA mismatch repair gene which has been identified by use of a method according to claims 1, 10 or 14.

19. An inactivating mutation in a variant of a DNA mismatch repair gene which has been identified by use of a method according to claims 1, 10 or 14.

20. A silent polymorphism in a variant of a DNA mismatch repair gene which has been identified by use of a method according to claims 1, 10 or 14.

21. A variant of the hMLH1 DNA mismatch repair gene which encodes an efficiency polymorphism selected from the group consisting of 217C (SEQ ID NO: 251), 265C (SEQ ID NO: 252), 265H (SEQ ID NO: 253), 326A (SEQ ID NO: 127), 62K (SEQ ID NO: 255), 69K (SEQ ID NO: 256), 25F (SEQ ID NO: 259), 25T (SEQ ID NO: 260), 28L (SEQ ID NO: 261), 64S (SEQ ID NO: 262), 111V (SEQ ID NO: 263), 262-del (SEQ ID NO: 264), 653R (SEQ ID NO: 265), 654L (SEQ ID NO: 266) and 659L (SEQ ID NO: 267).

22. A variant of the hMLH1 DNA mismatch repair gene which encodes an inactivating mutation selected from the group consisting of 44F (SEQ ID NO: 126), 44R (SEQ ID NO: 233), 44C (SEQ ID NO: 234), 44Q (SEQ ID NO: 235), 44H (SEQ ID NO: 236), 441 (SEQ ID NO: 237), 44L (SEQ ID NO: 238), 44P (SEQ ID NO: 239), 44T (SEQ ID NO: 240), 44W (SEQ ID NO: 241), 44Y (SEQ ID NO: 242), 44V (SEQ ID NO: 243), 67R (SEQ ID NO: 244), 68N (SEQ ID NO: 245), 102K (SEQ ID NO: 246), 107R (SEQ ID NO: 247), 117R (SEQ ID NO: 248), 542L (SEQ ID NO: 249), 659P (SEQ ID NO: 250), 82I (SEQ ID NO: 251), 84E (SEQ ID NO: 252) and 755W (SEQ ID NO: 280).

23. A variant of the hMLH1 DNA mismatch repair gene which encodes a silent polymorphism selected from the group consisting of 44A (SEQ ID NO: 232), 2191 (SEQ ID NO: 315), 219V (SEQ ID NO: 257), 681 T (SEQ ID NO: 258), 93G (SEQ ID NO: 271), 219L (SEQ ID NO: 272), 3261 (SEQ ID NO: 254), 663D (SEQ ID NO: 273), 718Y (SEQ ID NO: 274), 729V (SEQ ID NO: 275) and 751R (SEQ ID NO: 276).

24. A variant of the hMSH2 DNA mismatch repair gene which encodes an efficiency polymorphism selected from the group consisting of 322D (SEQ ID NO: 66) and 524P (SEQ ID NO: 277).
